(19) United States
(12) Patent Application Publication
(10) Pub. No.: US 2023/0017266 A1
(43) Pub. Date: Jan. 19, 2023

(54) Cyclooxygenase-2 Inhibition for the Treatment of SAA-high Asthma

(71) Applicant: THE USA, AS REPRESENTED BY THE SECRETARY, DEPT. OF HEALTH AND HUMAN SERVICES, Bethesda, MD (US)

(72) Inventors: Stewart J. Levine, North Potomac, MD (US); Xianglan Yao, Gaithersburg, MD (US); Maryann Kaler, Bethesda, MD (US); Alan T. Remaley, Bethesda, MD (US); Amisha V. Barochia, North Potomac, MD (US)

(73) Assignee: THE USA, AS REPRESENTED BY THE SECRETARY, DEPT. OF HEALTH AND HUMAN SERVICES, Bethesda, MD (US)

(21) Appl. No.: 17/781,530

(22) PCT Filed: Dec. 3, 2020
(86) PCT No.: PCT/US2020/063090

§ 371 Date: Jun. 1, 2022

Related U.S. Application Data
(60) Provisional application No. 62/943,087, filed on Dec. 3, 2019.

(51) Int. Cl. G01N 33/68; A61K 31/167; A61K 31/192; A61K 31/196; A61K 31/365; A61K 31/415; A61K 31/42; A61K 31/616; A61K 31/635; A61K 45/06; A61P 11/06

(52) U.S. Cl. CPC G01N 33/6893; A61K 31/167; A61K 31/192; A61K 31/196; A61K 31/365; A61K 31/415; A61K 31/42; A61K 31/616; A61K 31/635; A61K 45/06; A61P 11/06; G01N 2800/122; G01N 2800/52

(57) ABSTRACT

A method of treating an asthmatic subject is provided. The method includes determining serum level of serum amyloid A (SAA) in the subject; comparing the serum level with a pre-determined threshold; and administering to the subject a therapeutically effective amount of a COX-2 inhibitor if the serum level is greater than or equal to the threshold.

### CROSS-REFERENCE TO RELATED APPLICATION

This patent document claims priority from U.S. provisional patent application No. 62/943,087, filed Dec. 3, 2019, the entire disclosure of which is hereby incorporated by reference.

### FEDERALLY SPONSORED RESEARCH

This invention was made with government support under grant no. 1 ZIA HL006197 05 awarded by The National Institutes of Health. The government has certain rights in the invention.

### TECHNICAL FIELD

Disclosed herein are methods of treating an asthmatic condition associated with high serum level of serum amyloid A.

### BACKGROUND

Asthma is a chronic inflammatory disease effecting 14-15 million persons in the U.S. alone. Symptoms of asthma include recurrent episodes of wheezing, breathlessness, and chest tightness, and coughing, resulting from airflow obstruction. Airway inflammation associated with asthma can be detected through observation of a number of physiological changes, such as denudation of airway epithelium, collagen deposition beneath basement membrane, edema, mast cell activation, and inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, subbasement membrane fibrosis may occur, leading to persistent abnormalities in lung function.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and medacromil; long-acting bronchodilators, such as long-acting β 2-agonists and methylxanthines; and leukotriene modifiers. The quick-relief medications include short-acting β 2 agonists, anti-cholinergics, and systemic corticosteroids. There are many side effects associated with each of these drugs and none of the drugs alone or in combination is capable of preventing or completely treating asthma.

Thus, there is a need to develop new approaches to asthma treatment.

### SUMMARY OF THE INVENTION

An increased level of HDL-associated SAA in blood is associated with systemic inflammation and monocyte activation by a P2X7R/NF-κB/miR-155/COX2-dependent pathway. This suggests that a causal relationship might exist between SAA-mediated systemic inflammation and increased disease severity in SAA-high asthmatics. The treatment regimen of this patent document is based on the discovery of selective COX-2 inhibition for attenuation of HDL+SAA-mediated cytokine secretion by peripheral blood CD14⁺ monocytes.

An aspect of the patent document provides a method of treating an asthmatic subject. The method includes (i) determining serum level of serum amyloid A (SAA) in the subject; (ii) comparing the serum level with a pre-determined threshold; and (iii) administering to the subject a therapeutically effective amount of a COX-2 inhibitor if the serum level is greater than or equal to the threshold.

In some embodiments, the threshold is 95^{th} percentile of non-asthmatic subjects. In some embodiments, the threshold is about 100, about 104, about 108, about 110, about 115 or about 120 μg/ml. In some embodiments, the threshold is 90^{th} percentile of asthmatic subjects.

In some embodiments, the COX-2 inhibitor is an agent selected from the group consisting of acetylsalicylic acid (aspirin), 2-(4-isobutylphenyl)propanoic acid (ibuprofen), N-(4-hydroxyphenyl)ethanamide (paracetamol), (S)-6-methoxy-α-methyl-2-naphthaleneacetic acid (naproxen), 2-[(2,6-dichlorophenyl)amino] benzeneacetic acid (diclofenac), 4-[5-(4-methylphenyl)-3-(trifluoromethyl)-1H-pyrazol-1-yl] benzenesulfonamide (celecoxib), 4-[4-(methyl sulfonyl)phenyl]-3-phenyl-2(5H)-furanone (rofecoxib), and 445-Methyl-3-phenylisoxazol-4-yl)benzolsulfonamid (valdecoxib).

In some embodiments, the method includes administering the subject an additional agent for treating asthma.

In some embodiments, the method includes determining the subject as having higher than normal body-mass index (BMI), higher than normal serum C-reactive protein, or lower than normal serum IgE.

Another aspect provides a method of reducing inflammation associated with abnormal level of serum amyloid A (SAA) in a subject. The method includes (i) determining serum level of serum amyloid A (SAA) in the subject; (ii) comparing the serum level with a pre-determined threshold; and (iii) administering to the subject a therapeutically effective amount of a COX-2 inhibitor if the serum level is greater than or equal to the threshold.

In some embodiments, the threshold is 95^{th} percentile of non-asthmatic subjects. In some embodiments, the threshold is about 100, about 104, about 108, about 110, about 115 or about 120 μg μg/ml. In some embodiments, the threshold is 90^{th} percentile of asthmatic subjects.

In some embodiments, the subject has been diagnosed to have asthma. In some embodiments, the method further includes determining the subject as having higher than normal level of a cytokine. In some embodiments, the method further includes determining the subject as having higher than normal level of one or more cytokines selected from the group consisting of IL-6, IL-10, and TNF-α.

In some embodiments, the method further includes administering to the subject a P2X7R antagonist. In some embodiments, the method further includes determining the subject as having higher than normal body-mass index (BMI), higher than normal serum C-reactive protein, or lower than normal serum IgE.

Another aspect provides a method of reducing cytokines in a high SAA subject. The method includes (i) determining serum level of serum amyloid A (SAA) in the subject; (ii) comparing the serum level with a pre-determined threshold; and (iii) administering to the subject a therapeutically effective amount of one or more of COX-2 inhibitors, FPR2 inhibitors, P2X7R inhibitors and NF-κB antagonists. In some embodiments, the cytokines are selected from IL-6, TNF-α, IL-10, and IL-1β.

Another aspect provides a method of treating asthma in an subject in need thereof, wherein the subject has a serum SAA level of equal or greater than about 108 μg/ml. The method includes administering to the subject a therapeutically effective amount of a COX-2 inhibitor.

In some embodiments, the method further includes, prior to administering the COX-2 inhibitor, determining the subject as having higher than normal level of one or more cytokines selected from the group consisting of IL-6, IL-10, and TNF-α. In some embodiments, the method further includes, prior to administering the COX-2 inhibitor, determining the subject as having higher than normal body-mass index (BMI), higher than normal serum C-reactive protein, or lower than normal serum IgE.

Another aspect provides a method to identify an asthma subject suitable for treatment with COX-2 inhibitor. The method includes (i) determining serum level of serum amyloid A (SAA) in the subject; (ii) comparising the serum level with a pre-determined threshold; and (iii) identifying the subject as suitable for treatment with COX-2 inhibitor if the serum SAA level in the subject is greater than or equal to the threshold.

In some embodiments, the threshold is 95^{th} percentile of non-asthmatic subjects. In some embodiments, the threshold is 108.843 μg/ml. In some embodiments, the threshold is 90^{th} percentile of asthmatic subjects. In some embodiments, the method further includes determining the subject as having higher than normal level of one or more cytokines selected from the group consisting of IL-6, IL-10, and TNF-α.

### DESCRIPTION OF THE DRAWINGS

FIG. 1 illustrates enrichment of normal HDL with recombinant human SAA1. Western blots of normal HDL (Lanes 1 and 2), HDL that had been enriched with recombinant human SAA1 (Lanes 3 and 4), or recombinant human SAA1 alone (Lanes 5 and 6) were reacted with antibodies directed against SAA1, apolipoprotein A-I (APOA1), or paraoxonase-1 (PON1). The bottom panel is a gel stained with Coomassie G-250 to demonstrate equivalency of protein loading.

FIG. 2 illustrates that SAA-high asthma is characterized by older age, higher BMI, systemic inflammation, and increased asthma severity. a, Serum SAA levels in non-asthmatic (n=154) and asthmatics (n=146). The dashed line indicates the upper 95^{th} centile value of serum SAA (>108.8 μg/ml) in the non-asthmatic group (n=153 after exclusion of the extreme outlier who subsequently developed giant cell arteritis and polymyalgia rheumatica), which was used as the threshold to define SAA-high versus SAA-low asthmatics. Comparison between SAA-high (n=16) and SAA-low asthmatics (n=130) regarding; b, age, c, body mass index (BMI), d, serum C-reactive protein, e, serum IgE, f-h, prevalence of obesity (BMI>30 kg/m²), inhaled corticosteroid (ICS) use, and severe asthma, i, serum IL-6, j, prevalence of IL-6 high asthma, k, serum TNF-α, and l, serum IL-10. For a-l, Two group comparisons (asthmatic vs non-asthmatic) or (SAA-high vs. SAA-low) of continuous variables were analyzed by t-test, taking into account the equality of their variances. Categorical values were analyzed using a chi-square test, except when counts were <5, in which case the Fisher's exact test was used. m, Comparison between the SAA-high asthmatics (n=16) and asthmatics with the lowest serum SAA levels (n=16) regarding the concentration of SAA and APOA1 in HDL isolated from plasma. n, HDL isolated from plasma (4 μg of protein/ml) from the SAA-high asthmatic group (n=16) and asthmatics with the lowest serum SAA levels (n=16) was used to stimulate THP-1 monocytes for 24 h and the amount of IL-1β, IL-6, TNF-α and IL-10 secreted into the medium was quantified. A representative result of 3 independent experiments is shown. For m and n, differences between groups were analyzed using the Mann-Whitney test.

FIG. 3 illustrates that HDL enriched with serum amyloid A (HDL+SAA1) signals via P2X7R to induce the secretion of IL-1β, IL-6, TNF-α and IL-10 by CD14+ monocytes. a, Ex vivo cultures of classical CD14⁺/CD16⁻, non-classical CD14^{dim}/CD16⁺, and intermediate CD14⁺/CD16⁺ monocytes from asthmatics were isolated by flow cytometry and cultured with media (control), plasma from the monocyte donor (4 μg of protein/ml), normal HDL (4 μg of protein/ml), or HDL enriched with recombinant human SAA1 (HDL+SAA1) (4 μg of protein/ml) for 24 h. In addition, classical CD14⁺/CD16⁻ monocytes were stimulated with HDL enriched with the amount of lipopolysaccharide (0.01 ng/ml) present in recombinant human SAA1 (HDL+LPS). n=20 subjects, except for n=11 subjects in the HDL+LPS group. Pairwise comparisons of all groups vs. media (control), one-way ANOVA with Dunnett's multiple comparisons test. b and c, To characterize the receptors involved in HDL+SAA1 signaling, ex vivo cultures of CD14⁺ monocytes isolated from healthy volunteers by flow cytometry were cultured with media (control), normal HDL, or HDL+SAA1 for 24 h. CD14⁺ monocytes were also pre-treated with, b, the FPR2 antagonist, WRW4 (40 μM), c, the P2X7R antagonist, A438079 (10 or vehicle (water or DMSO) for 1 h prior to stimulation with HDL+SAA1 for 24 h. n=9 subjects, HDL+SAA1 plus WRW4 or A438079 vs. HDL+SAA1 plus vehicle, Wilcoxon matched-pairs signed rank test.

FIG. 4 illustrates that HDL enriched with serum amyloid A (HDL+SAA1) induces cytokine secretion from CD14+ monocytes via a P2X7R/NF-κB/COX-2-dependent pathway. a, Volcano plot showing differentially expressed mRNA transcripts from classical CD14+/CD16− monocytes from asthmatics (n=10) that were stimulated ex vivo with or without HDL enriched with recombinant human SAA1 (HDL+SAA1). Additional experiments utilized ex vivo cultures of CD14⁺ monocytes isolated by flow cytometry from healthy subjects to characterize the HDL+SAA1-mediated P2X7R/NF-κB/COX-2-dependent pathway, except where indicated. b, PTGS2 mRNA levels were quantified by qRT-PCR in cells stimulated with normal HDL or HDL+SAA1 and presented as RQ (relative quantification). n=7 subjects, pairwise comparisons of all groups vs. media, repeated measures one-way ANOVA with Dunnett's multiple comparisons test. c, Western blot showing COX-1 and COX-2 protein in cells stimulated with normal HDL or HDL+SAA1. GAPDH is shown as a control for equivalency of protein loading. The Western blot shown is representative of 3 independent experiments using cells from different subjects. Densitometry is presented as the ratio of COX-1 or COX-2 to GAPDH. n=3 subjects, pairwise comparisons of all groups vs. media, repeated measures one-way ANOVA with Dunnett's multiple comparisons test. d, PGE2 and TXB2 secreted by cells cultured with HDL+SAA1 alone or HDL+SAA1 plus celecoxib (50 μM), diclofenac (1 mM), or vehicle (DMSO or water, respectively) for 24 h. n=5, HDL+SAA1 plus celecoxib or diclofenac vs. HDL+SAA1 plus vehicle (DMSO or water), Mann-Whitney test. The result shown is representative of 4 independent experiments using cells from different subjects. e, Histogram overlay plot showing serine 529 phosphorylation of the NF-κB p65 subunit in CD45⁺/CD14⁺/CD16⁻ monocytes following 15 minutes of stimulation with HDL+SAA1 with or without A438079 (10 μM). This histogram is representative of 4 independent experiments using cells from different subjects. f, PTGS2 mRNA levels in cells cultured with HDL+SAA1 with or without the P2X7R antagonist, A438079 (10 μM), or vehicle (DMSO) for 24 h. n=10 subjects, HDL+SAA1 plus A438079 vs. HDL+SAA1 plus DMSO, Wilcoxon matched-pairs signed rank test. g, PTGS2 mRNA levels in cells cultured with HDL+SAA1 with or without BAY-11-7082 (10 μM), TPCA-1 (1 μM), or vehicle (DMSO) for 24 h. n=6, HDL+SAA1 plus BAY-11-7082 or TPCA-1 vs. HDL+SAA1 plus DMSO, one-way ANOVA with Dunnett's multiple comparisons test. The result shown is representative of 5 independent experiments using cells from different subjects. h, Cytokines secreted by cells cultured with HDL+SAA1 with or without BAY-11-7082 (10 .iM), TPCA-1 (1 μM), or vehicle (DMSO) for 24 h. n=6, HDL+SAA1 plus BAY-11-7082 or TPCA-1 vs. HDL+SAA1 plus DMSO, one-way ANOVA with Dunnett's multiple comparisons test. The result shown is representative of 3 independent experiments using cells from different subjects. i and j, Cytokines secreted by cells cultured with HDL+SAA1 alone or HDL+SAA1 plus, i, celecoxib (50 μM), j, diclofenac (1 mM), or vehicle (DMSO or water, respectively) for 24 h. n=5, HDL+SAA1 plus celecoxib or diclofenac vs. HDL+SAA1 plus vehicle (DMSO or water), Mann-Whitney test. The result shown is representative of 4 independent experiments using cells from different subjects.

FIG. 5 illustrates raw data for COX-1 Western blots. Western blot showing COX-1 and GAPDH protein in CD14⁺ monocytes stimulated with normal HDL or HDL+SAA1.

FIG. 6 illustrates raw data for COX-2 Western blots. Western blot showing COX-2 and GAPDH protein in CD14⁺ monocytes stimulated with normal HDL or HDL+SAA1.

FIG. 7. HDL+SAA1 induces cytokine secretion by CD14⁺ monocytes via miR-155. a, miR-155-5p levels were quantified by qRT-PCR in CD14⁺ monocytes stimulated with normal HDL or HDL+SAA1 and presented as RQ (relative quantification). N=9 asthmatic subjects, pairwise comparisons of all groups vs. media, repeated measures one-way ANOVA with Dunnett's multiple comparisons test. Additional experiments utilized ex vivo cultures of CD14⁺ monocytes isolated by flow cytometry from healthy subjects to characterize the role of miR-155-5p in the HDL+SAA1 signaling pathway. b, miR-155-5p levels in CD14⁺ monocytes cultured with HDL+SAA1 with or without the P2X7R antagonist, A438079 (10 or vehicle (DMSO) for 24 h. n=11 subjects, HDL+SAA1 plus A438079 vs. HDL+SAA1 plus DMSO, Wilcoxon matched-pairs signed rank test. c, miR-155-5p levels in CD14⁺ monocytes cultured with HDL+SAA1 with or without BAY-11-7082 (10 TPCA-1 (1 or vehicle (DMSO) for 24 h. n=6, HDL+SAA1 plus BAY-11-7082 or TPCA-1 vs. HDL+SAA1 plus DMSO, one-way ANOVA with Dunnett's multiple comparisons test. The result shown is representative of 5 independent experiments using cells from different subjects. d, PTGS2 mRNA levels in CD14⁺ monocytes cultured with HDL+SAA1 with or without the miR-155-5p antagonist, or negative control, for 3 days. n=6, HDL+SAA1 plus miR-155-5p antagonist vs. HDL+SAA1 plus negative control, Mann-Whitney test. The result shown is representative of 3 independent experiments using cells from different subjects. e, Cytokines secreted by cells cultured with or without the miR-155-5p antagonist, or negative control, for 3 days. n=6, HDL+SAA1 plus miR-155-5p antagonist vs. HDL+SAA1 plus negative control, Mann-Whitney test. The result shown is representative of 3 independent experiments using cells from different subjects.

FIG. 8 illustrates gating strategy for isolation of human monocyte subsets from peripheral blood mononuclear cells by flow cytometry. Contour plots showing the gating strategy used to identify debris-free, single, live, SSC^{moderate/high,} CD45⁺ cells from which CD14⁺/CD16″ (classical), CD14⁺/CD16⁺ (intermediate), CD14^{dim}/CD16⁺ (non-classical), and CD14⁺ (classical and intermediate) monocytes were sorted or analysed.

FIG. 9 illustrates that THP-1 monocytes stimulated with endogenous SAA-high HDL induces IL-1β, IL-6, and TNF-α secretion that could be inhibited by both the FPR2 antagonist, WRW4, as well as the P2X7R antagonist, A438079.

FIG. 10 illustrates that inhibitors of NF-κB signaling pathways, BAY 11-7082 and TPCA1, abrogate the ability of endogenous SAA-high HDL to induce cytokine secretion by THP-1 monocytes.

FIG. 11 illustrates that Celecoxib (10 nM) inhibits endogenous SAA-high HDL-induced increases in IL-1β, IL-6, and TNF-α secretion by THP-1 monocytes.

### DETAILED DESCRIPTION

This patent document discloses a causal relationship between serum amyloid A (SAA) mediated systemic inflammation and increased disease severity in SAA-high asthmatics. The approach of COX-2 inhibition provides a targeted treatment for conditions associated with abnormal levels of SAA, especially in certain asthmatic subjects.

HDL mediates reverse cholesterol transport out of cells to reduce atherosclerosis and attenuate inflammation. HDL may also have a protective effect in asthma based upon an association with less severe airflow obstruction. SAA is an acute-phase response protein that is synthesized by the liver during inflammation and is secreted into the blood where it binds to HDL. This converts HDL from a protective, anti-inflammatory particle to a dysfunctional, pro-inflammatory form. SAA in the lung also drives inflammation in asthma. Bronchoalveolar lavage fluid (BALF) SAA levels are increased in severe asthmatics and correlate with higher numbers of BALF neutrophils. Furthermore, an endotype of neutrophil-predominant severe asthma is characterized by high BALF levels of SAA and low BALF levels of lipoxin A₄ (LXA₄) that induces IL-8 expression by lung epithelial cells that had been stably transfected to express the formyl peptide receptor 2 (FPR2), which is also known as the ALX receptor. Instillation of SAA into murine lungs increases BALF levels of multiple pro-inflammatory cytokines, including IL-13, IL-6, and TNF-α, while mice sensitized by oropharyngeal administration of ovalbumin plus SAA to the lungs develop steroid-resistant allergic inflammation.

While the following text may reference or exemplify specific embodiments of agent or use thereof, it is not intended to limit the scope of the agent or its use to such particular reference or examples. Various modifications may be made by those skilled in the art, in view of scientific and practical considerations, such as replacement of the substituent and treatment of different diseases.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “about” refers to the referenced numeric indication plus or minus 10% of that referenced numeric indication.

The term “agent” refers to any compound or molecule capable of eliciting a response in a biological system such as, for example, living cell(s), tissue(s), organ(s), and being(s). Biologically active agents can include natural and/or synthetic agents. Thus, an agent is intended to be inclusive of any substance intended for use in the diagnosis, cure, mitigation, treatment, or prevention of disease or in the enhancement of desirable physical or mental development and conditions in a subject.

The term “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably.

The term “subject” and “patient” are used interchangeably and refer to humans or animals including for example sheep, horses, cattle, pigs, dogs, cats, rats, mice, birds, and reptiles. Preferably, the subject is a human or other mammal.

The term “effective amount” or “therapeutically effective amount” refers to an amount that is sufficient to ameliorate, or in some manner reduce a symptom or stop or reverse progression of a condition associated with high serum level of serum amyloid A. Such amount may be administered as a single dosage or may be administered according to a regimen, whereby it is effective.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

An aspect of the disclosure provides a method of treating a subject diagnosed to have asthma, comprising:
(i) determining serum level of serum amyloid A (SAA) in the subject;
(ii) comparising the serum level with a pre-determined threshold; and
(iii) administering to the subject a therapeutically effective amount of a COX-2 inhibitor if the serum level is greater than or equal to the threshold.

SAA is secreted into the blood where it resides on high-density lipoprotein (HDL) particles. Remodeling of HDL in blood by binding SAA can convert it to a dysfunctional particle that induces systemic inflammation and increases disease severity in asthma. SAA-high asthma is often characterized by more severe disease, older age, obesity, increased systemic inflammation, and higher serum levels of cytokines including IL-6, TNF-α, and IL-10. It has been discovered that endogenous HDL isolated from SAA-high asthmatics have a higher SAA content and induced augmented cytokine secretion by monocytes as compared to endogenous HDL isolated from asthmatics with the lowest serum SAA levels. For instance, HDL enriched with SAA1 induces the secretion of IL-1β, IL-6, TNF-α, and IL-10 from CD14⁺ monocytes via a P2X7R/NF-κB/miR-155/COX-2 pathway. COX-2 inhibition can attenuate the cytokine secretion resulting from the enriched SAA1.

The pre-determined threshold is based on the evaluation of a defined population, which can be a group of non-asthmatic subjects or a group of asthmatic subjects. The group needs to include a sufficient number of people in order to yield a value of statistical significance. As illustrated in the example below, a threshold of a certain percentile (x %) based on a group of non-asthmatic subjects is a value higher than the SAA levels of this particular percentage of the subjects in the group. In other words, only the remaining subjects (1-x %) of this group have SAA levels higher than this value. In some embodiments, the threshold is 50^{th} percentile, 55^{th} percentile, 60^{th} percentile, 65^{th} percentile, 70^{th} percentile, 75^{th} percentile, 80^{th} percentile, 85^{th} percentile, 90^{th} percentile, 95^{th} or 98^{th} percentile of a defined population. In some embodiments, the defined population is a group of non-asthmatic subjects. In some embodiments, the defined population is a group of asthmatic subjects. In some embodiments, the threshold is 95^{th} percentile of SAA levels from non-asthmatic subjects. In some embodiments, the threshold is 90^{th} percentile of SAA levels from asthmatic subjects.

In some embodiments, the threshold is about 100, about 104, about 108, about 110, about 115 or about 120 μg/ml. In some embodiments, the threshold is 108.843 μg/ml.

Various COX-2 inhibitors can be used for the methods of this patent document. Non-limiting examples include acetylsalicylic acid (aspirin), 2-(4-isobutylphenyl)propanoic acid (ibuprofen), N-(4-hydroxyphenyl)ethanamide (paracetamol), (S)-6-methoxy-α-methyl-2-naphthaleneacetic acid (naproxen), 2-[(2,6-dichlorophenyl)amino] benzeneacetic acid (diclofenac), 4-[5-(4-methylphenyl)-3-(trifluoromethyl)-1H-pyrazol-1-yl] benzenesulfonamide (celecoxib), 4-[4-(methylsulfonyl)phenyl]-3-phenyl-2(5H)-furanone (rofecoxib), and 4-(5-Methyl-3-phenylisoxazol-4-yl)benzolsulfonamid (valdecoxib).

The methods disclosed herein may include the administration of a second agent for treating asthma. Non-limiting examples of the secondary agent include beta2-adrenoceptor agonists (SABA, e.g. salbutamol), macrolide antibiotics (e.g., azithromycin), anticholinergic medications (e.g. ipratropium), adrenergic agonists (e.g. inhaled epinephrine), corticosteroids, long-acting beta-adrenoceptor agonists (LABA) (e.g. salmeterol and formoterol), leukotriene receptor antagonists (e.g. montelukast and zafirlukast), mast cell stabilizers (e.g. cromolyn sodium), vitamin C and vitamin E. Additional agents or treatment include omalizumab, mepolizumab, reslizumab, benralizumab, dupilumab and bronchial thermoplasty. The secondary agent may be administered simultaneously, sequentially, or at any disarable interval under the direction of a qualified professional or medical doctor.

Besides older age, obesity, more severe asthma, and increased systemic inflammation, SAA-high asthmatics are often characterized by abnormal levels of one or more additional biomarkers. In some embodiments, the methods disclosed herein also include determining the subject as having higher than normal body-mass index (BMI), higher than normal serum C-reactive protein, or lower than normal serum IgE. The normal levels of BMI, serum C-reactive protein, and serum IgE can be easily determined from healthy subjects and used for comparison with the subjects to be tested.

Another aspect of the patent document provides a method of reducing inflammation associated with abnormal level of serum amyloid A (SAA) in a subject. The method includes:
(i) determining serum level of serum amyloid A (SAA) in the subject;
(ii) comparing the serum level with a pre-determined threshold; and
(iii) administering to the subject a therapeutically effective amount of a COX-2 inhibitor if the serum level is greater than or equal to the threshold.

The scope of COX-2 inhibitors is as described above. In some embodiments, the subject is asthmatic. The optional additional agents for treating athma are as described above. In some embodiments, the subject is nonasthmatic. Non-asthmatic subjects may be defined by a history and physical examination that is negative for asthma, plus the absence of airway hyperreactivity based upon a negative methacholine bronchoprovocation challenge. Asthmatic subjects may be defined using NHLBI guidelines (Guidelines for the diagnosis and management of asthma: full report 2007, (U. S Dept. of Health and Human Services, National Institutes of Health, National Heart, Lung, and Blood Institute, Bethesda, Md., 2010)). Severe asthma may be defined using ERS/ATS guidelines (International ERS/ATS guidelines on definition, evaluation and treatment of severe asthma. Eur Respir J 43, 343-373 (2014)).

As demonstrated in the examples below, endogenous HDL from SAA-high asthmatics has increased SAA content and is more pro-inflammatory than endogenous HDL isolated from asthmatics with the lowest serum SAA levels. Monocyte activation has been reported to promote systemic inflammation in obese asthmatics. Here, it has been discovered that remodeling of the HDL proteome with increased SAA activates peripheral blood monocytes to secrete cytokines that are increased in the serum of SAA-high asthmatics. Administration of COX-2 to a subject with higher than the threshold SAA can thus control and reduce inflammation in the subject.

Abnormal levels of certain cytokines may also indicate inflammation associated with high SAA in a subject. Therefore in some embodiments, the methods include determining the subject as having higher than normal level of one or more cytokines. Non-limiting examples of the cytokines include IL-1β, IL-6, IL-8, IL-10, IL-17A, and TNF-α. In some embodiments, the methods include identifying the subject as having higher than normal levels of IL-6, IL-10, and TNF-α.

Other factors relevant to high levels of SAA include body-mass index (BMI), higher than normal serum C-reactive protein, and lower than normal serum IgE. Accordingly in some embodiments, the methods include analyzing one or more biomarkers and determining the subject as having higher than normal body-mass index (BMI), higher than normal serum C-reactive protein, and/or lower than normal serum IgE.

Because high SAA levels also activate NF-κB signaling pathways downstream of P2X7R to increase PTGS2 mRNA levels and pro-inflammatory cytokine secretion, the methods disclosed herein may also include administering one or more additional agents including for example FPR2 inhibitors, P2X7R inhibitors and NF-κB antagonists. By attenuating HDL+SAA-mediated phosphorylation of p65 NF-κB and controlling increases in PTGS2 mRNA, secretion of cytokines (e.g. IL-6, TNF-α, IL-10, IL-1β by CD14⁺ monocytes or THP-1 monocytes) can be reduced. For instance, it has been discovered that the P2X7R antagonist (A438079) inhibited IL-1 β secretion by THP-1 monocytes stimulated with endogenous HDL from SAA-high asthmatics.

Non-limiting examples of FPR2 inhibitors include 4-butoxy-N-[2-(4-hydroxyphenyl)-4-oxo-1,2-dihydroquinazolin-3-yl]benzamide (quin-C7), isopropylureido-FLFLF, (5R)-4-(Cyclohexylmethyl)-1-[(2R)-1-[(2S)-2- [[(6S)-2,3-dioxo-6-propan-2-ylpiperazin-1-yl]methyl]pyrrolidin-1-yl]-3-naphthalen-2-ylpropan-2-yl]-5-[(4-hydroxyphenyl)methyl]piperazine-2,3-dione (compound 1754-31), WRWWWW (PubChem SID 135652639), t-Boc-FLFLF (PubChem SID 135652599) PBP10, BOC2 or WRW4.

Non-limiting examples of NF-κB antagonists include BAY-11-7082, TPCA1, zanubrutinib, acalabrutinib, ibrutinib, dasatinib, tirabrutinib, rilzabrutinib, evobrutinib, orelabrutinib, ABBV-105, ABBV-599, SAR-442168, branebrutinib, TAS-5315, remibrutinib, BMS-986142, fenebrutinib, poseltinib, spebrutinib, spebrutinib, DTRMWXHS-12, CT-1530, REDX08608, M-7583, ARQ-531, vecabrutinib, TAK-020, BIM068, AC-0058TA, SN-1011, BIM-091, TG-1701, CG-806, PF-06650833, CA-4948, R-835, BAY-1834845, BAY-1830839, birinapant, APG-1387, LCL-161, ASTX660, Debio 1143, and CUDU-427.

Non-limiting examples of P2X7R inhibitors/antagonists include the following.

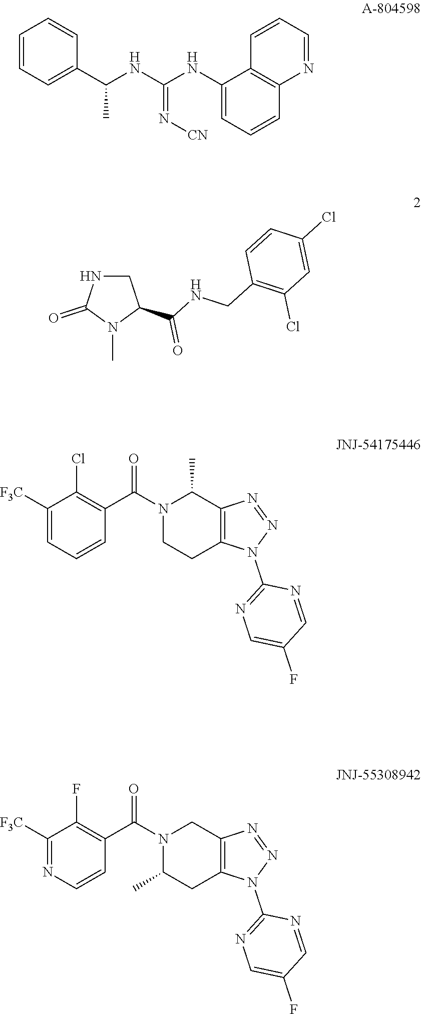 [SMILES: C[C@@H](N/C(=N/C#N)Nc1cccc2ncccc12)c1ccccc1 ; CN1C(=O)NC[C@H]1C(=O)NCc1ccc(Cl)cc1Cl ; C[C@@H]1c2nnn(-c3ncc(F)cn3)c2CCN1C(=O)c1cccc(C(F)(F)F)c1Cl ; C[C@H]1Cc2c(nnn2-c2ncc(F)cn2)CN1C(=O)c1ccnc(C(F)(F)F)c1F]

The COX-2 inhibitors, FPR2 inhibitors, P2X7R inhibitors, and NF-κB antagonists can be used, alone or in any combination thereof, with or without additional agents for treating asthma, in any of the methods disclosed in this patent document. For instance, a method of this patent document may include administering one, two, three, or four of a COX-2 inhibitor, an FPR2 inhibitor, a P2X7R inhibitor and an NF-κB antagonist, with or without an additional asthma treatment agent. In some embodiments, the method includes administering a COX-2 inhibitor optionally in combination with one, two, three of an FPR2 inhibitor, a P2X7R inhibitor and an NF-κB antagonist, with or without an additional asthma treatment agent.

A related method is the reduction of cytokines in a high SAA subject. As illustrated in the examples, cytokine secretion induced by SAA-high HDL involves a series of events including for example NF-κB signaling pathways downstream of P2X7R to increase PTGS2 mRNA levels. One or more inhibitors against one or more of the involved stages can be administered to the subject to reduce cytokine secrection. Non-limiting examples include COX-2 inhibitors, FPR2 inhibitors, P2X7R inhibitors and NF-κB antagonists. In some embodiments, the cytokines are selected from IL-6, TNF-α, IL-10, and IL-1β. The method includes (i) determining serum level of serum amyloid A (SAA) in the subject; (ii) comparing the serum level with a pre-determined threshold; and (iii) administering to the subject a therapeutically effective amount of one or more of the above mentioned inhibitors and/or antagonists. This method is also suitable for controlling or reducing cytokine storm in the above-identified high SAA subjects. In some embodiments, the method includes diagnosing the subjects as having a disease or condition commonly associated with a “cytokine storm.” Non-limiting examples of such diseases or conditions include COVID-19 infection, sepsis, systemic inflammatory response syndrome (SIRS), cachexia, septic shock syndrome, traumatic brain injury (e.g., cerebral cytokine storm), graft versus host disease (GVHD), or the result of treatment with activated immune cells, e.g., IL-2 activated T cells, T cells activated with anti-CD19 Chimeric Antigen Receptor (CAR) T cells.

Another aspect provides a method of treating asthma in a subject in need thereof, wherein the subject has a serum SAA level of equal or greater than pre-determined threshold. The method includes administering to the subject a therapeutically effective amount of a COX-2 inhibitor. Optionally, an additional asthma treatment agent can be administered. The scope of COX-2 inhibitor and the optional asthma treatment agent is as described above. Further, one or more additional agents including for example the above described FPR2 inhibitors, P2X7R inhibitors and NF-κB antagonists may also be administered.

In some embodiments, the method includes prior to administering the COX-2 inhibitor, determining the subject as having higher than normal level of one or more cytokines. In some embodiments, the cytokines are selected from IL-6, IL-10, and TNF-α. In some embodiments, the method includes, prior to administering the COX-2 inhibitor, determining the subject as having higher than normal body-mass index (BMI), higher than normal serum C-reactive protein, or lower than normal serum IgE.

For any of the methods disclosed herein, the scope of the pre-determined threshold can be as described above. In some embodiments, the threshold is 95^{th} percentile of SAA levels from non-asthmatic subjects. In some embodiments, the threshold is about 100, about 104, about 108, about 110, about 115 or about 120 μg/ml. In some embodiments, the threshold is 108.843 μg/ml. In some embodiments, the threshold is 90^{th} percentile of SAA levels from asthmatic subjects.

Another aspect provides a method to identify an asthma subject suitable for treatment with COX-2 inhibitor. By gauging the administration of the COX-2 inhibitor to the serum level of SAA, a more targeted and effective therapy can be achieved. The method includes:
(i) determining serum level of serum amyloid A (SAA) in the subject;
(ii) comparing the serum level with a pre-determined threshold; and
(iii) identifying the subject as suitable for treatment with COX-2 inhibitor if the serum SAA level in the subject is greater than or equal to the threshold.

The scope of the pre-determined threshold and the COX-2 inhibitors are as described above. In some embodiments, threshold is 95^{th} percentile of SAA levels from non-asthmatic subjects. In some embodiments, the threshold is about 100, about 104, about 108, about 110, about 115 or about 120 μg/ml. In some embodiments, the threshold is 108.843 μg/ml. In some embodiments, the threshold is 90^{th} percentile of SAA levels from asthmatic subjects.

In some embodiments, the method further includes determining the subject as having higher than normal level of one or more cytokines. In some embodiments, the subject has higher than normal level of one or more cytokines selected from IL-6, IL-10, and TNF-α.

Another aspect of the patent document provides a kit containing one or more agents such as COX-2 inhibitors, asthma medications, and P2X7R antagonist. These agents may be formulated by any method well known in the art and may be prepared for administration by any route, including, without limitation, parenteral, oral, sublingual, transdermal, topical, intranasal, intratracheal, or intrarectal. The kit also includes a manual for practicing the methods disclosed herein. A database containing the pre-determined threshold and other standards of normal biomarkers such as cytokines can also be included. The database can be stored in a computer-readable medium coupled to one or more data processing apparatus. The kit may further include tools or equipments for collecting samples and testing the levels of the biomarkers.

In non-human animal studies, applications of the agents disclosed herein are commenced at higher dosage levels, with dosage being decreased until the desired effect is no longer achieved adverse side effects disappear. The dosage may range broadly, depending upon the desired effects and the therapeutic indication. Typically, dosages may be about 10 microgram/kg to about 100 mg/kg body weight, preferably about 100 microgram/kg to about 10 mg/kg body weight. Alternatively dosages may be based and calculated upon the surface area of the patient, as understood by those of skill in the art.

The exact formulation, route of administration and dosage for the agents (e.g. COX-2 inhibitors) can be chosen by the individual physician in view of the patient's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the agent(s) thereof administered to the subject or patient can be from about 0.5 to about 1000 mg/kg of the patient's body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

Although the exact dosage of an agent will be determined on a drug-by-drug basis, in most cases, some generalizations regarding the dosage can be made. The daily dosage regimen for an adult human patient may be, for example, an oral dose of about 0.1 mg to 2000 mg of the agent (e.g. COX-2 inhibitor), preferably about 1 mg to about 500 mg, e.g. 5 to 200 mg. In other embodiments, an intravenous, subcutaneous, or intramuscular dose of the active ingredient of about 0.01 mg to about 100 mg, preferably about 0.1 mg to about 60 mg, e.g. about 1 to about 40 mg is used. Alternatively the agent may be administered by continuous intravenous infusion, preferably at a dose of up to about 1000 mg per day. As will be understood by those of skill in the art, in certain situations it may be necessary to administer the agent disclosed herein in amounts that exceed, or even far exceed, the above-stated, preferred dosage range in order to effectively and aggressively treat particularly aggressive diseases or infections. In some embodiments, the agent will be administered for a period of continuous therapy, for example for a week or more, or for months or years.

The agents disclosed herein can be evaluated for efficacy and toxicity using known methods. For example, the toxicology of the agent may be established by determining in vitro toxicity towards a cell line, such as a mammalian, and preferably human, cell line. The results of such studies are often predictive of toxicity in animals, such as mammals, or more specifically, humans. Alternatively, the toxicity in an animal model, such as mice, rats, rabbits, or monkeys, may be determined using known methods. The efficacy of a particular agent may be established using several recognized methods, such as in vitro methods, animal models, or human clinical trials. Recognized in vitro models exist for nearly every class of condition. Similarly, acceptable animal models may be used to establish efficacy of therapeutic agents to treat such conditions. When selecting a model to determine efficacy, the skilled artisan can be guided by the state of the art to choose an appropriate model, dose, and route of administration, and regime. Of course, human clinical trials can also be used to determine the efficacy of an agent in humans.

The agents disclosed herein may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert.

All references cited herein are incorporated herein in their entireties.

The following examples serve to further illustrate the present disclosure.

### Examples

Study Participants. Subjects provided written informed consent to participate in protocols 96-H-0100 and/or 99-H0076, which were approved by institutional review board of the National Heart, Lung, and Blood Institute (NHLBI). The asthmatic (n=146) and non-asthmatic (n=154) cohort was evaluated between 1999 and 2016. Non-asthmatic subjects were defined by a history and physical examination that was negative for asthma, plus the absence of airway hyperreactivity based upon a negative methacholine bronchoprovocation challenge. Asthmatic subjects were defined using NHLBI guidelines, whereas severe asthma was defined using ERS/ATS guidelines. Asthmatic subjects had a history and physical exam consistent with asthma, plus either reversible airflow obstruction after inhalation of a short-acting β2-agonist or airway hyperreactivity based upon a positive methacholine bronchoprovocation challenge test. Asthmatic (n=26) and healthy (n=58) research subjects also served as peripheral blood donors between 2017 and 2019 for experiments characterizing the HDL+SAA1-mediated signaling pathway in monocytes.

Serum Analysis. Analyses were performed on non-fasting blood samples that were stored at −80° C. The CLIA-certified NIH Clinical Research Center Clinical Laboratory performed the standard laboratory tests and serum lipid profiles. Serum SAA levels were quantified using the human SAA ELISA kit (Thermo Scientific, Frederick, Md.), serum IL-6 and IL-17A were quantified using the human Quantikine high-sensitivity ELISA kits (R&D Systems, Minneapolis, Minn.)²³ and data were acquired using a SpectraMax M2 spectrophotometer (Molecular Devices, San Jose, Calif.). Serum levels of IL-1β, IL-8, IL-10, and TNF-α were quantified using the V-PLEX human cytokine immunoassay system and data were acquired using a QuickPlex SQ 120 instrument (Mesoscale Scale Discovery, Rockville, Md.).

HDL Isolation and Analysis. HDL was isolated from plasma by size-exclusion chromatography using two Superose 6 columns in series on an Akta FPLC system (GE Healthcare Life Sciences, Pittsburgh, Pa.), as previously described. FPLC fractions were assayed for cholesterol content (Cholesterol E, catalog #996021611, FUJIFILM Wako Diagnostics USA, Mountain View, Calif.) to identify HDL fractions, which were pooled and concentrated using a 10-kDa exclusion filter (Amicon Ultra Centrifuge Filters, Millipore, Darmstadt, Germany). The content of SAA in purified HDL was quantified using the human SAA ELISA kit, while APOA1 was quantified using the human APOA1 ELISA development kit (Mabtech, Nacka Strand, Sweden). THP-1 monocytes (American Type Culture Collection, Manassas, Va.) were treated with HDL from SAA-high asthmatics (n=16), as well as HDL from asthmatics with the lowest SAA levels (n=16), and the quantity of IL-13, IL-6, TNF-α, and IL-10 secreted into culture medium over 24 h was assayed using Human DuoSet ELISA kits (R&D Systems, Minneapolis, Minn.) and data were acquired using a SpectraMax M2 spectrophotometer (Molecular Devices, San Jose, Calif.).

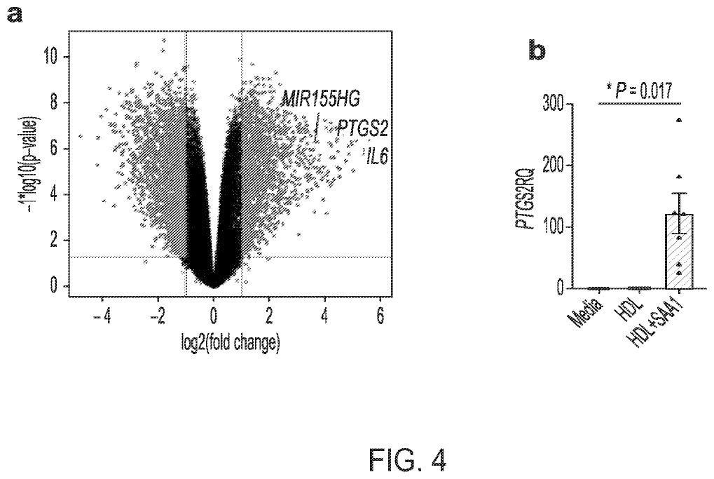

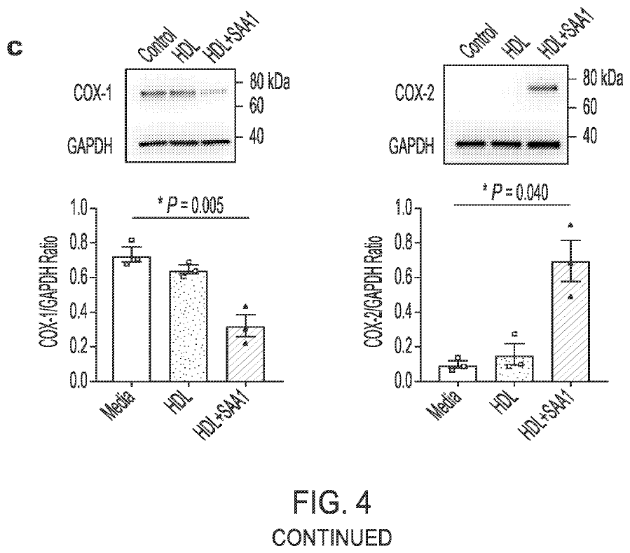

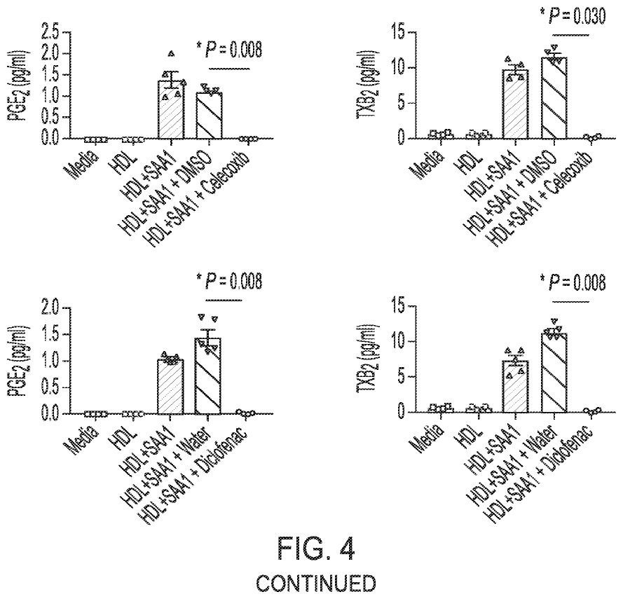

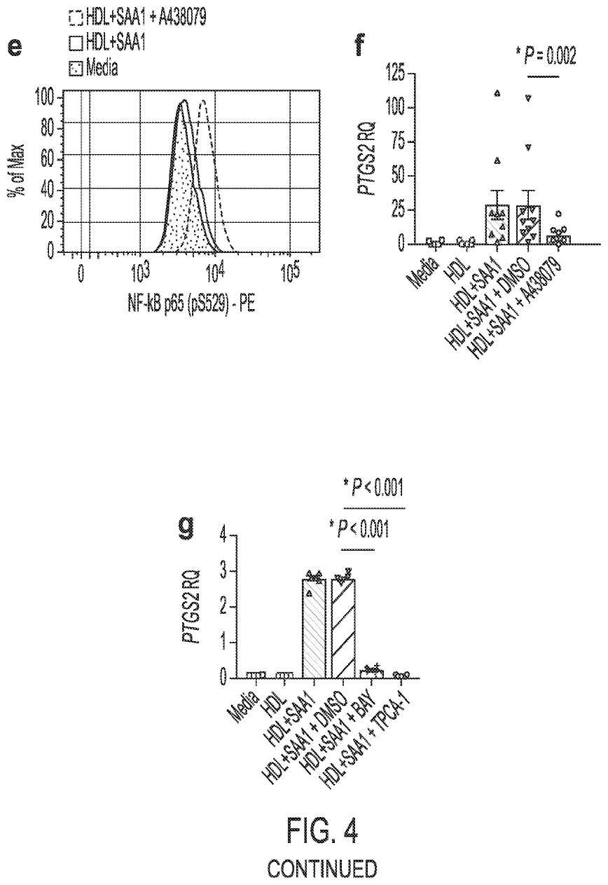

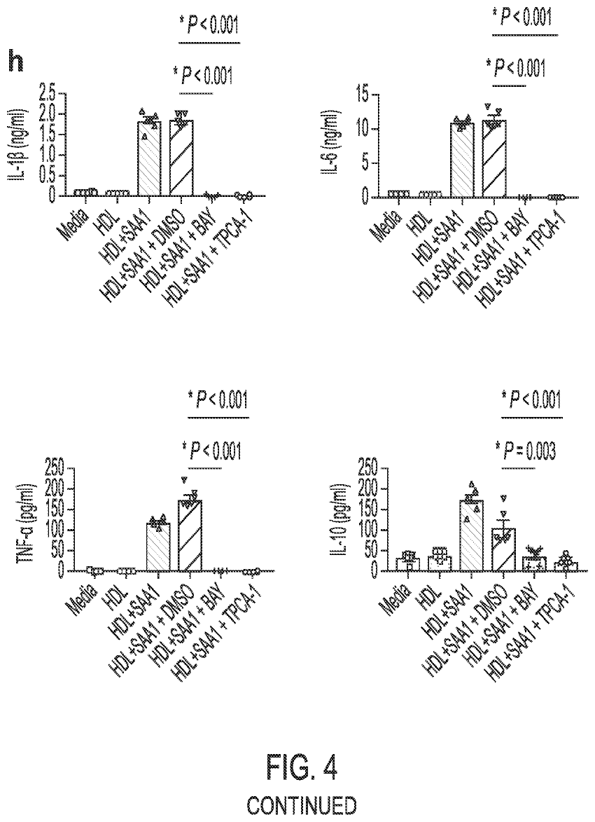

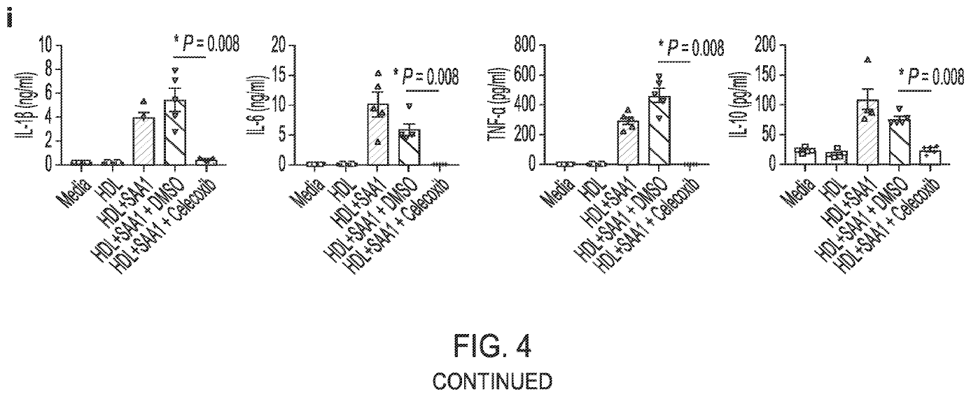

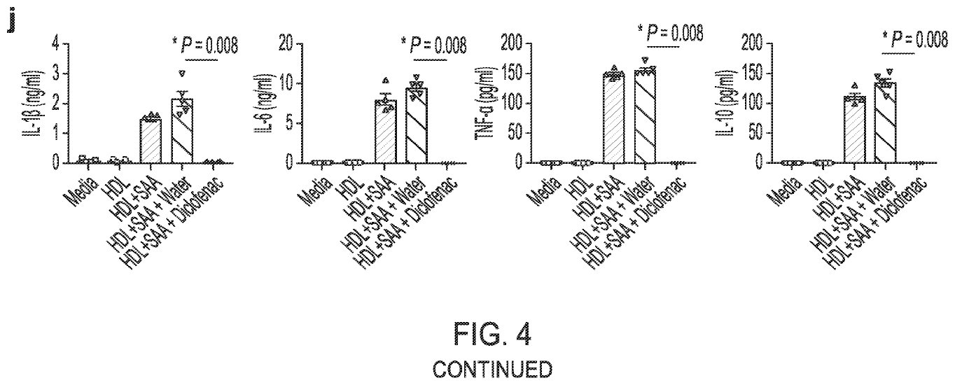

Isolation of Peripheral Blood Monocytes. Peripheral blood monocytes were isolated using a two-step process. Peripheral blood was diluted with PBS at a 1:1 ratio, slowly overlaid onto 30 ml of Lymphoprep™ (catalog #07811, StemCell Technologies, Vancouver Canada) in a 50 ml conical tube, and centrifuged at 400×g for 25 min without braking. The peripheral blood mononuclear cell (PBMC) layer was carefully transferred to another 50 ml conical tube, washed with PBS, followed by two washes with FACS buffer (0.5 mM EDTA, 1% BSA, and 1% mouse serum in PBS). Monocytes subsets were identified and sorted by flow cytometry using the following antibodies: PE mouse anti-human CD45 clone HI30 (catalog #555483), BV421 mouse anti-human CD14 clone MφP9 (catalog #563743), and FITC mouse anti-human CD16 clone B73.1 (catalog #561308), and a FACS ARIA Fusion sorter equipped with 355, 407, 488, 532 and 640 nm LASER lines using FACS DIVA 8.0 software (all from BD Biosciences, San Jose, Calif.). The gating strategy identified a population of debris-free, single, live, ^{SSCmoderate/high}, CD45⁺ cells from which CD14⁺/CD16⁻ (classical), CD14⁺/CD16⁺ (intermediate), CD14^{dim}/CD16⁺ (non-classical), and CD14⁺ (classical and intermediate) cells were sorted and utilized for experiments, as indicated (Supplemental FIG. 4).

HDL+SAA1 Stimulation of Peripheral Blood Monocytes Subsets. Purified monocyte subsets were cultured in (RPMI-1640 media with 2% fetal bovine serum) for 24 h with either plasma from the monocyte donor as a control, normal HDL alone, or normal HDL enriched with recombinant human serum amyloid A-1 that had a LPS content of 0.01 ng/ig (Peprotech, Rocky Hill, N.J.). As previously described, normal HDL was isolated from plasma obtained from healthy subjects by sequential KBr differential gradient ultracentrifugation at 330,000 g and extensive dialysis with 50 mM HEPES, 50 mM NaCL, 5 mM MgCl₂ and 2 mM CaCl₂, pH 7.0, at 4° C. Normal HDL was complexed with recombinant human serum amyloid A-1 at a 2:1 ratio by mixing and incubated at RT overnight to generate HDL+SAA1, which was filtered using a 100-kDa exclusion filter (Amicon Ultra Centrifuge Filters, Millipore, Darmstadt, Germany) to remove any uncomplexed SAA1. Normal HDL was also complexed with the amount of lipopolysaccharide (catalog # L4391, E. coli 0111:B4, y-irradiated, MilliporeSigma, St. Louis, Mo.) present in recombinant human SAA1. Additionally, classical CD14⁺ monocytes were treated with the P2X7R antagonist, A438079, the IKK inhibitor, BAY-11-7082, the IKKβ (IKK-2) inhibitor, TPCA-1, or the selective COX-2 inhibitor, celexocib, all from MilliporeSigma(St. Louis, Mo.). Cells were also treated with the FPR2 antagonist, WRW4, or diclofenac from Tocris (Minneapolis, Minn.). The amount of IL-1β, IL-6, TNF-α and IL-10 secreted into cell culture supernatants was quantified using human DuoSet ELISA kits (R&D Systems) and data were acquired using a SpectraMax M2 spectrophotometer (Molecular Devices, San Jose, Calif.). Western blotting, as previously described⁴³, was performed using antibodies directed against COX-1 (catalog #160110) and COX-2 (catalog #160112), both from Cayman Chemical (Ann Arbor, Mich.). Equivalency of protein loading was established using an antibody directed against GAPDH (catalog #MAB5718) from R&D Systems (Minneapolis, Minn.). Western blots images were captured using an iBright FL1000 Western Blot Imaging System (ThermoFisher Scientific, Waltham, Mass.) and quantified using NIH ImageJ software (imagej.nih.gov).

RNA-seq Analysis of Classical CD14+/CD16− Monocytes. Classical CD14+/CD16− monocytes were isolated from the blood of asthmatic subjects by negative selection using the RosetteSep Human Monocyte Enrichment Cocktail (#15068, StemCell Technologies), followed by flow sorting. Purified CD45⁺/CD14⁺/CD16⁻ classical monocytes were cultured for 24 h in RPMI 1640 media+2% fetal bovine serum with normal HDL that had or had not been enriched with recombinant human SAA1. Total RNA was purified using the Direct-Zol™ RNA MiniPrep kit (catalog #R2052; Zymo Research, Irvine, Calif.) and sequencing libraries were constructed from 100 ng to 500 ng of total RNA with the TruSeq Stranded Total RNA Library Prep kit (catalog #20020596; Illumina, San Diego, Calif.) and the Ribo-Zero™ rRNA Removal (catalog #MRZH11124; Illumina, San Diego, Calif.) kit. Fragment sizes of the RNAseq libraries were verified using a 2100 Bioanalyzer (#G2939BA; Agilent Technologies, Santa Clara, Calif.) and concentrations were quantified using a Qubit 3 Fluorometer (#Q33226; ThermoFisher Scientific, Waltham, Mass.). Libraries were loaded onto a HiSeq 3000 Sequencing System (#SY-401-3001; Illumina, San Diego, Calif.) and 2×75 bp paired-end read sequencing was performed. Fastq files were produced using bcl2fastq Conversion Software v2.20 (Illumina, San Diego, Calif.).

Rigorous quality controls of paired-end reads were assessed using FastQC tools. If required, adapter sequences and low-quality bases were trimmed using Cutadapt. Reads were aligned to the reference genome using the latest version of STAR, which is a splice-aware aligner that sequentially aligns reads to the known transcriptome and genome. FeatureCounts was used for gene level abundance estimation using the GENCODE (ν25) comprehensive gene annotations. Principal component analysis (PCA) was used to assess outlier samples. Genes were kept in the analysis if they had raw read counts>5 in at least half the samples.

Differential expression analysis comparing cases versus controls at the gene levels of summarization were then carried out using open source Limma R package. Limma-voom, was employed to implement a gene-wise linear modelling which processes the read counts into log 2 counts per million (log CPM) with associated precision weights. The log CPM values were normalized between samples using trimmed mean of M-values (TMM). Features with q<5% were declared as genome-wide significant.

RNA-seq data are available on the Gene Expression Omnibus website (https://www.ncbi.nlm.nih.gov/geo/query/acc.cgi?acc=GSE134591) under accession # GSE134591.

Quantitative RT-PCR (qRT-PCR). Total RNA was purified using the Direct-Zol™ RNA MiniPrep kit and total RNA (100 ng) was reverse transcribed into cDNA using the High-Capacity cDNA Reverse Transcription Kit (Applied Biosystems, Foster City, Calif.). Polymerase chain reactions (PCR) were performed on duplicate cDNA samples using TaqMan Universal PCR Master Mix, FAM-MGB dye-labeled Taqman® PTGS2 probe (Assay ID Hs00153133_m1, catalog #4331182), and a 7900 Real Time PCR System running Sequence Detector version 2.4 software, all from Applied Biosystems (Foster City, Calif.). Gene expression was quantified relative to expression of 18S rRNA using the control sample as a calibrator to calculate the difference in Ct values (ΔΔCt) and presented as relative mRNA expression. For quantification of miR-155-5p expression, miRNA present in total RNA (10 ng) was converted to cDNA using the microRNA cDNA Reverse Transcription kit (Applied Biosystems, Foster City, Calif.). Polymerase chain reaction (PCR) was performed on duplicate cDNA samples using TaqMan Universal PCR Master Mix, Taqman® MicroRNA Assay for has-miR-155-5p (Assay ID #002623, catalog #4427975), and a 7900HT Fast Real-Time PCR System running Sequence Detector version 2.4 software (ThermoFisher Scientific, Waltham, Mass.). miR-155-5p expression was quantified relative to expression of U6 snRNA using the control sample as calibrator to calculate the difference in Ct values (ΔΔCt) and presented as relative mRNA expression.

Phosphorylation of NF-κB p65. PBMCs isolated using Lymphoprep™ were resuspended in X-VIVO™ 15 serum-free hematopoietic cell medium (Lonza, Walkersville, Md.) and treated with the P2X7R antagonist, A438079 (10 ∝M), for 2 h prior to stimulation with HDL+SAA1 for 15 min⁵³. Subsequent cell processing was performed at 4° C. or on ice. Cells were washed with cold PBS, centrifuged at 300×g for 5 min, and reacted in a volume of 100 ∝l PBS for 15 min with the following antibodies; APC-Cy™7 mouse anti-human CD45 clone 2D1 (catalog #557833), FITC mouse anti-human CD14 clone M5E2 (catalog #555397), APC mouse anti-human CD16 clone B73.1 (catalog #561304), all form BD Biosciences (San Jose, Calif.) and cell viability dye (L34959, ThermoFisher Scientific, Frederick, Md.). Cells were washed with 1 ml of cold PBS to remove excess antibodies, centrifuged at 300×g for 5 min, fixed with 100 μL Cytofix Fixation Buffer (catalog #554655, BD Biosciences, San Jose, Calif.) on ice for 10 min, and washed with cold Phosflow Perm Buffer III (catalog#558050, BD San Jose, Calif.). Cells were then reacted with PE mouse anti-human NF-κB p65 antibody (pS529) (clone K10-895.12.50) (catalog #558423, BD Biosciences, San Jose, Calif.), which recognizes phosphorylated serine 529, in Phosflow Perm Buffer III for 30 min on ice. Cells were washed with Phosflow Perm Buffer III and PBS, resuspended in PBS, and analyzed using a Fortessa analyzer equipped with 355, 407, 488, 532 and 640 nm LASER lines using FACS DIVA 8.0 software (BD Biosciences, San Jose, Calif.). CD14⁺ monocytes were identified as CD45⁺/SSC^{hi}/CD14⁺/CD16⁻ cells. Mean fluorescence intensity (MFI) was calculated and overlay histogram plots were generated to quantify phosphorylation of NF-κB p65 serine 529 among various treatment conditions.

miR-155 Inhibition. CD14+ monocytes were suspended in 4D human monocyte Nucleofector™ solution (Amaxa™ P3 Primary Cell 4D-Nucleofector™ X kit, catalog #V4XP-3024, Lonza, Walkersville, Md.) to a final concentration of 2×10⁶ cells/100 jtl at room temperature. 2×10⁶ cells were mixed with miR-155-5p inhibitor (50 ∝M) (MH12601, catalog#4464084) or miRNA inhibitor negative control #1 (catalog#4464076), both from ThermoFisher Scientific (Frederick, Md.), transferred to a Nucleocuvette™ Vessel, and transfection was performed using a 4D-Nucleofector™ X Unit (Lonza, Walkersville, Md.). Cells were then cultured in RPMI-1640 medium with 2% fetal bovine serum, with or without HDL+SAA1. CD14⁺ monocytes were plated at a density of 4×10⁴ cells/well in a 96-well plate to collect culture media for cytokine quantification and the remaining cells were plated in a 24-well plate to isolate total RNA for qRT-PCR. After 72 h, cytokine secretion was quantified by ELISA and mRNA levels were quantified by qRT-PCR.

Statistical Analyses. Continuous variables, in clinical data, were assessed for normality using visual inspection of their distribution. Non-normally distributed variables were log-transformed before performing the analyses. Extreme outlier threshold was defined as mean+/−3 standard deviations which corresponds to the 99.7 percentile in a normal distribution. Pearson correlation was used to assess associations between approximately normal variables. Group comparisons (asthmatics vs. non-asthmatics) or (SAA-high vs. SAA-low) of continuous variables are based on a t-test, taking into account the equality of their variances. For the categorical variables a chi-square test was used, unless we had cells with expected counts less than 5, in which case we used the Fisher's exact test. Experimental data were analyzed by Mann-Whitney test, Wilcoxon matched-pairs signed rank test, and ordinary or repeated measures one-way ANOVA with Dunnett's multiple comparisons test, as indicated in the figure legends. Principal component analysis was used to assess outlier samples. P<0.05 was considered significant and all tests were two-sided. Data were analyzed using SAS 9.4 (SAS institute, Cary, N.C.) and GraphPad Prism software (version 7.0b; GraphPad Software, La Jolla, Calif.).

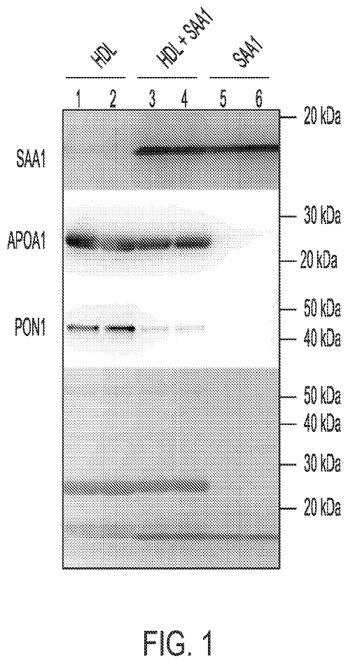

Without being bound by any particular theory, it is hypothesized that remodeling of HDL in blood by binding SAA might convert it to a dysfunctional particle that induces systemic inflammation and increases disease severity in asthma. In the analysis of a cohort of 154 non-asthmatic and 146 asthmatic subjects (Table 1), it was found that serum SAA levels were significantly higher in asthmatics (FIG. 1a). One non-asthmatic, who later developed giant cell arteritis and polymyalgia rheumatica, was considered to be an extreme outlier (serum SAA value>mean+3 standard deviations) and was excluded from the cohort of non-asthmatics (n=153) that was subsequently analyzed to establish the upper 95th centile value of serum SAA as >108.8 tg/m123. This threshold was used to characterize 11% of the asthmatic cohort as having SAA-high asthma, which was associated with older age, higher BMI and serum C-reactive protein (a biomarker of systemic inflammation), lower serum IgE (a biomarker of allergic sensitization), as well as an increased prevalence of obesity, inhaled corticosteroid use, and severe asthma (FIG. 1b-1h). In contrast, there were no significant differences between SAA-high and SAA-low asthmatics regarding airflow obstruction, peripheral blood eosinophil or neutrophil counts, and the prevalence of hypertension, diabetes, or use of prednisone or lipid-lowering medications (Table 2).

TABLE 1

| | Non-asthmatic (n = 154) | | Asthmatic (n = 146) | | P value |
|---|---|---|---|---|---|
| Age (years) | 34.5 ± 13.6 | | 39 ± 15.2 | | 0.007 |
| Gender (female/male) | 99 (64%)/55 (36%) | | 97 (66%)/49 (34%) | | ns |
| Ethnicity | | | | | <0.001 |
| African American | 29 | (19%) | 41 | (28%) | |
| Asian | 24 | (16%) | 10 | (7%) | |
| White | 101 | (66%) | 86 | (59%) | |
| Other | 0 | (0%) | 9 | (6%) | |
| BMI (kg/m²) | 25.6 | (22.4-29.1) | 26.1 | (23.2-32.2) | 0.003 |
| Obese (BMI > 30 kg/m²) | 28 | (18%) | 46 | (32%) | 0.007 |
| Hypertension | 4 | (3%) | 20 | (14%) | <0.001 |
| Diabetes | 1 | (1%) | 2 | (1%) | ns |
| Spirometry | | | | | |
| FEV₁ (% predicted) | 109.5 ± 14 | | 85.3 ± 18.7 | | <0.001 |
| FVC (% predicted) | 103.1 ± 12.9 | | 95.2 ± 14.7 | | <0.001 |
| FEV₁/FVC | 83.9 ± 5.8 | | 71.0 ± 11.8 | | <0.001 |
| Serum IgE (IU/ml)¹ | 39 | (11-115) | 212 | (71-472) | <0.001 |
| Blood Eosinophils (cells/μl) | 119 | (70-169) | 222 | (125-346) | <0.001 |
| Blood Neutrophils (K/μl) | 3.6 ± 1.1 | | 3.9 ± 1.6 | | 0.030 |
| Serum C-reactive protein (mg/L) ² | 1 | (0.5-2.5) | 1.3 | (0.6-4.4) | 0.004 |
| Total Cholesterol (mg/dL) ³ | 177.5 ± 34 | | 186.9 ± 35 | | 0.021 |
| HDL-C (mg/dL) ³ | 50.7 ± 17.5 | | 55.5 ± 17.3 | | 0.019 |
| APOA1 (mg/dL) ³ | 164.3 ± 31.0 | | 166.4 ± 31.3 | | ns |
| LDL-C (mg/dL) ^{3, 4} | 102.1 ± 29.9 | | 107.4 ± 31.7 | | ns |
| Triglycerides (mg/dL) ³ | 126.5 ± 91.3 | | 120.2 ± 63.5 | | ns |
| lipid-lowering medication utilization | 8 | (5%) | 5 | (3%) | ns |
| Serum Amyloid A (μg/mL) | 13.7 | (6.3-26.8) | 18.1 | (7.5-42.4) | 0.010 |

*Data are presented as mean + SD or number (percentage). Comparisons of non-asthmatic versus asthmatic subjects were performed using a t-test, taking into account the equality of their variances, for continuous variables, or a chi-square or Fisher's exact test for categorical variables.
*Comparisons of BMI, serum IgE, blood eosinophil counts, serum C-reactive protein, serum triglycerides, and serum amyloid A were performed using log-transformed values. Data for these values are presented as median (interquartile range).
¹Quantification of serum IgE was not performed on 5 non-asthmatic subjects.
² Quantification of serum C-reactive protein was not performed on 2 non-asthmatic and 9 asthmatic subjects.
³ Quantification of total cholesterol, HDL-C, APOA1 and triglycerides were not performed on 2 non-asthmatic and 3 asthmatic subjects.
⁴ LDL-C could not be calculated for 3 non-asthmatic and 1 asthmatic subjects due to serum triglyceride levels >400 mg/dl.

TABLE 2

| | SAA-low (n = 130) | | SAA-high (n = 16) | | P value |
|---|---|---|---|---|---|
| Gender (female/male) | 85 (65%)/45 (35%) | | 12 (75%)/4 (25%) | | ns |
| Ethnicity | | | | | ns |
| African American | 36 | (27%) | 5 | (31%) | |
| Asian | 10 | (8%) | 0 | (0%) | |
| White | 76 | (59%) | 10 | (63%) | |
| Other | 8 | (6%) | 1 | (6%) | |
| Hypertension | 17 | (13%) | 3 | (19%) | ns |
| Diabetes | 2 | (2%) | 0 | (0%) | ns |
| Lipid-lowering Medication Use | 3 | (2%) | 2 | (13%) | ns |
| Oral Corticosteroid Use | 3 | (2%) | 1 | (6%) | ns |
| Spirometry | | | | | |
| FEV₁ (% predicted) | 86.1 ± 18.5 | | 80.3 ± 19.4 | | ns |
| FVC (% predicted) | 95.5 ± 14.4 | | 93.8 ± 17.6 | | ns |
| FEV₁/FVC | 71.6 ± 11.9 | | 67.5 ± 10.4 | | ns |
| Blood Eosinophils (cells/μl) | 222 | (131-343) | 220 | (105-462) | ns |
| Blood Neutrophils (K/μl) | 3.8 ± 1.5 | | 4.5 ± 1.5 | | ns |
| Total Cholesterol (mg/dL) ¹ | 187.4 ± 34.1 | | 182.1 ± 43.2 | | ns |
| Serum HDL-C (mg/dL) ¹ | 54.9 ± 17.3 | | 60.7 ± 16.8 | | ns |
| Serum APOA1 (mg/dL) ¹ | 166.3 ± 31.2 | | 167.0 ± 33.5 | | ns |
| Serum LDL-C (mg/dL) ^{1, 2} | 108.2 ± 30.8 | | 100.8 ± 39.0 | | ns |
| Serum Triglycerides (mg/dL) ¹ | 105 | (72-161) | 93 | (67-123) | ns |
| Serum IL-1β (pg/ml) | 0 | (0-0) | 0 | (0-0) | ns |
| Serum IL-8 (pg/ml) | 10.2 | (7.7-14.1) | 13.1 | (9.2-15.6) | ns |
| Serum IL-17A (pg/ml) | 0 | (0-0) | 0 | (0-0) | ns |

Data are presented as mean + SD or number (percentage). Comparisons of SAA-high versus SAA-low asthmatic subjects were performed using a t-test, taking into account the equality of their variances, for continuous variables, or a chi-square or Fisher's exact test for categorical variables.
Comparisons of blood eosinophil counts, serum triglycerides, serum IL-1□, serum IL-8, and serum IL-17A were performed using log-transformed values. Data for these values are presented as median (interquartile range).
¹ Quantification of total cholesterol, HDL-C, APOA1 and triglycerides were not performed 1 SAA-high asthmatic and 2 SAA-low asthmatics.
² LDL-C could not be quantified for 1 SAA-low subject due to a triglyceride level >400 mg/dl.

It was then assessed whether serum levels of cytokines associated with obesity, such as IL-13, IL-6, IL-8, and TNF, as well as IL-17A, were modified in SAA-high asthmatics. The study also quantified IL-10, which is primarily considered to be an anti-inflammatory cytokine, but also has context-dependent pro-inflammatory properties. As shown in FIGS. 1i and 1j, both serum IL-6 and the prevalence of IL-6-high asthma were significantly increased in SAA-high asthmatics as compared to SAA-low asthmatics. Serum TNF-α and IL-10 were also significantly increased in SAA-high asthmatics (FIGS. 1k and 1l). There were no differences in serum levels of IL-13, IL-8, or IL-17A, however, IL-13 and IL-17A were measurable in only 9% and 23% of subjects, respectively, which precluded a definitive determination as to whether levels are modified in SAA-high asthmatics (Table 2).

Since monocyte activation has been reported to promote systemic inflammation in obese asthmatics, It was investigated whether remodeling of the HDL proteome with increased SAA activates peripheral blood monocytes to secrete cytokines that were increased in the serum of SAA-high asthmatics (IL-6, TNF-α, IL-10), as well as IL-1β. Endogenous HDL isolated from plasma of SAA-high asthmatics (n=16) had an increased content of SAA as compared to HDL isolated from the 16 asthmatics with the lowest serum SAA levels, whereas there was no significant difference in APOA1 (FIG. 1m). Treatment of THP-1 monocytes with endogenous HDL isolated from SAA-high asthmatics induced greater increases in cytokine secretion than endogenous HDL isolated from SAA-low asthmatics (FIG. 1n). Thus, endogenous HDL from SAA-high asthmatics has increased SAA content and is more pro-inflammatory than endogenous HDL isolated from asthmatics with the lowest serum SAA levels.

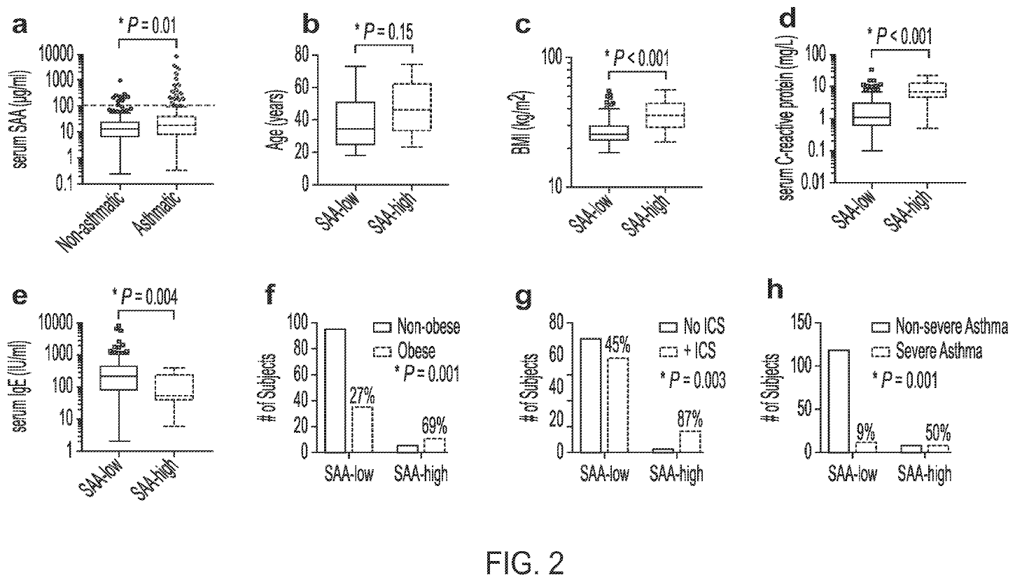

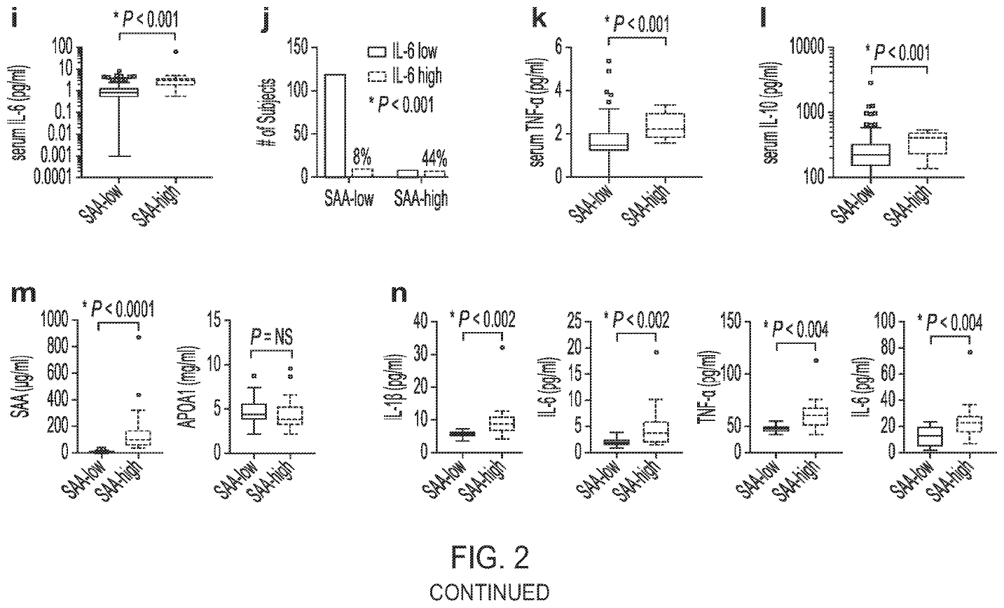

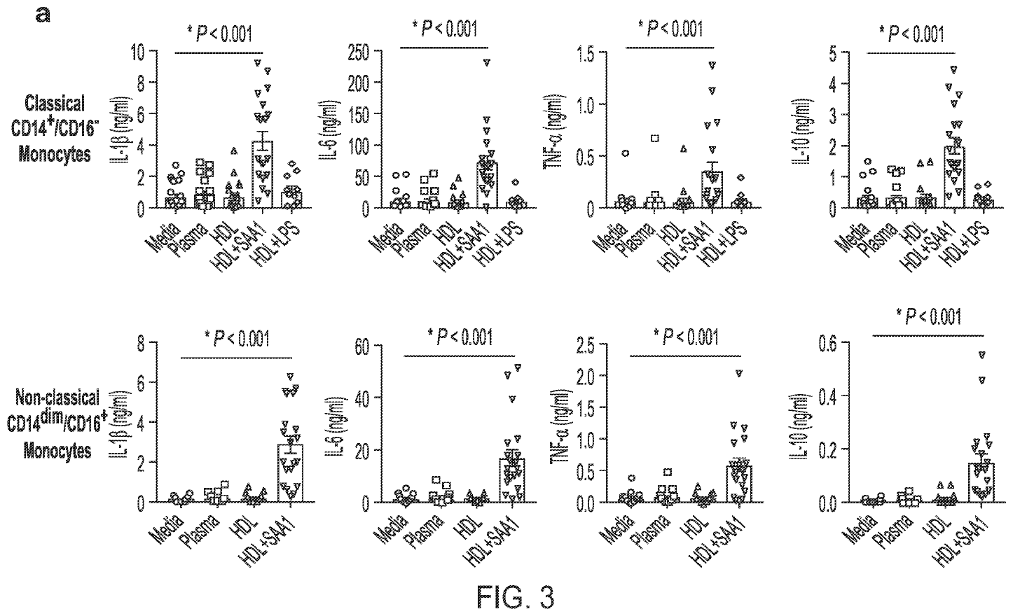

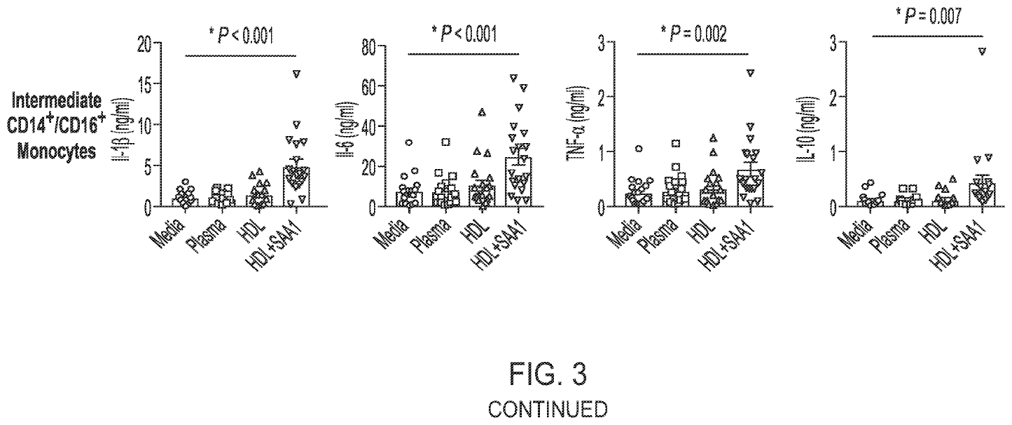

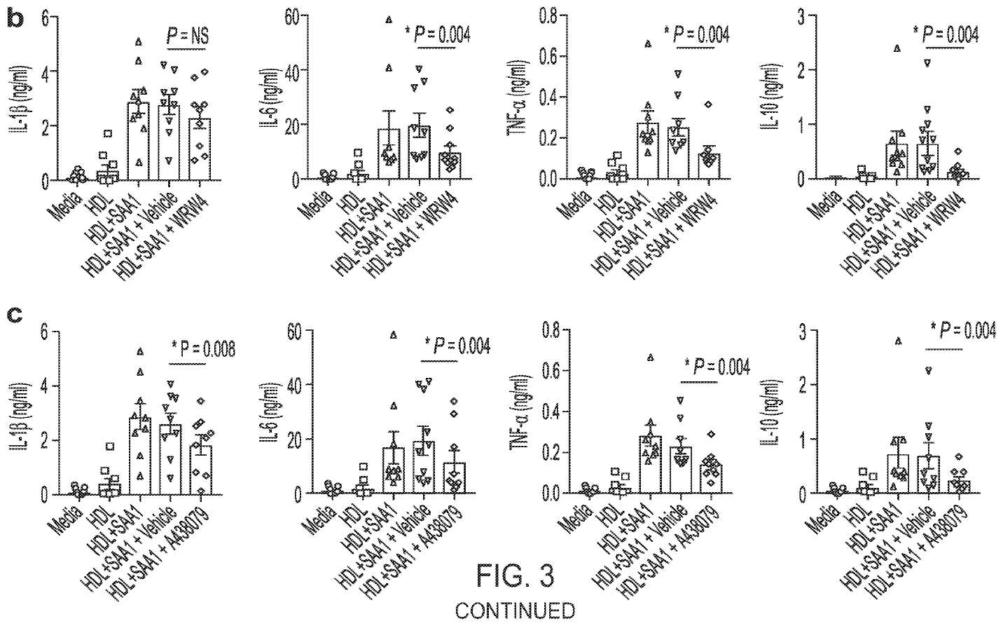

Next, a model system was developed to characterize the mechanism by which HDL-associated SAA induces cytokine secretion by monocytes. Incubation of normal human HDL with recombinant human SAA1, hereafter referred to as HDL+SAA1, markedly increased the SAA1 content of HDL, which was associated with a reduction in the anti-oxidant enzyme, paraoxonase 1 (PON1) (FIG. 2). Classical (CD14⁺/CD16⁻), non-classical (CD14^{dim}/CD16⁺) and intermediate (CD14⁺/CD16⁺) monocyte subsets were isolated from the blood of asthmatics and cultured with plasma from the same donor as a control, normal HDL alone, or HDL+SAA1. As shown in FIG. 3a, HDL+SAA1 promoted IL-10, IL-6, TNF-α, and IL-10 secretion from all three monocyte subsets. In contrast, plasma or normal HDL alone did not modify cytokine secretion by any monocyte subset. HDL complexed with the amount of lipopolysaccharide (LPS) present in recombinant human SAA did not increase cytokine production by classical CD14⁺/CD16⁻ monocytes. Although SAA can interact with multiple receptors, its pro-inflammatory effects on human monocytes are primarily mediated by FPR2. It was found that the FPR2 antagonist, WRW4, significantly attenuated the HDL+SAA1-mediated secretion of IL-6, TNF-α and IL-10, but not IL-1β, by CD14⁺ monocytes (FIG. 3b). In contrast, the P2X7R antagonist, A438079, significantly reduced HDL+SAA1-mediated secretion of all four cytokines. Therefore, the study focused on characterizing the P2X7R-mediated pathway that regulates HDL+SAA1-induced secretion of IL-1β, IL-6, TNF-α, and IL-10 by CD14⁺ monocytes.

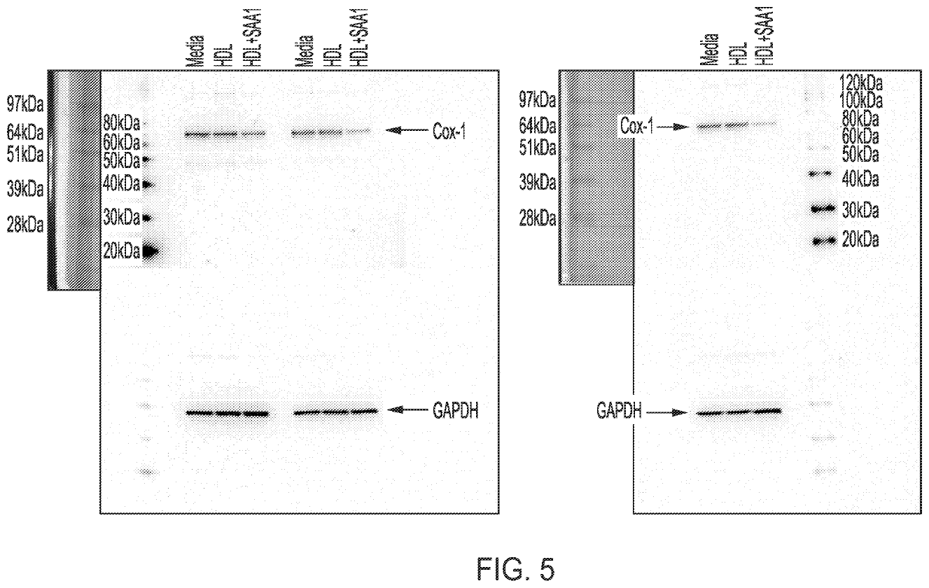

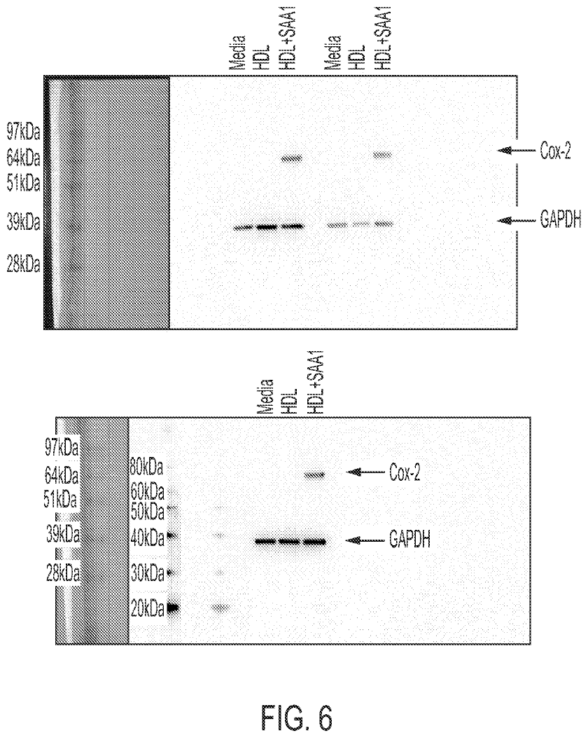

A RNA-seq analysis of classical CD14⁺/CD16⁻ monocytes isolated from asthmatics and stimulated with HDL+SAA1 identified the two most highly up-regulated mRNA transcripts as IL6 and PTGS2 (prostaglandin-endoperoxide synthase 2), which encodes cyclooxygenase-2 (COX-2) (FIG. 4a). Consistent with the RNA-seq results, HDL+SAA1 increased both PTGS2 mRNA (FIG. 4b) and COX-2 protein expression by CD14⁺ monocytes, whereas cyclooxygenase-1 (COX-1) protein was decreased (FIG. 5 and FIG. 6). Furthermore, HDL+SAA1 induced significant increases in products of the COX-2 biosynthetic pathway, prostaglandin E₂ and thromboxane B₂ (an inactive metabolite of thromboxane A2), which were suppressed by the selective COX-2 inhibitors, celecoxib and diclofenac (FIG. 4d). Since SAA has been reported to activate NF-κB signaling in human monocytes and P2X7R can signal via NF-κB, it was investigated whether HDL+SAA1 activates NF-κB signaling pathways downstream of P2X7R to increase PTGS2 mRNA levels and pro-inflammatory cytokine secretion. It was shown that the P2X7R antagonist, A438079, attenuated both HDL+SAA1-mediated phosphorylation of p65 NF-κB (FIG. 4e) and increases in PTGS2 mRNA (FIG. 4f). In addition, inhibitors of NF-κB signaling, BAY 11-7082 and TPCA1, prevented HDL+SAA1-mediated increases in PTGS2 mRNA (FIG. 4g) and cytokine secretion (FIG. 4h). The ability of HDL+SAA1 to markedly induce COX-2 expression in CD14⁺ monocytes also implied that HDL+SAA1-mediated cytokine secretion might be COX-2-dependent. As shown in FIGS. 4i and 4j, the selective COX-2 inhibitors, celecoxib and diclofenac, significantly inhibited HDL+SAA1-mediated secretion of IL-13, IL-6, TNF-α, and IL-10 by CD14⁺ monocytes, which suggests that COX-2 inhibition might attenuate systemic inflammation in SAA-high asthmatics.

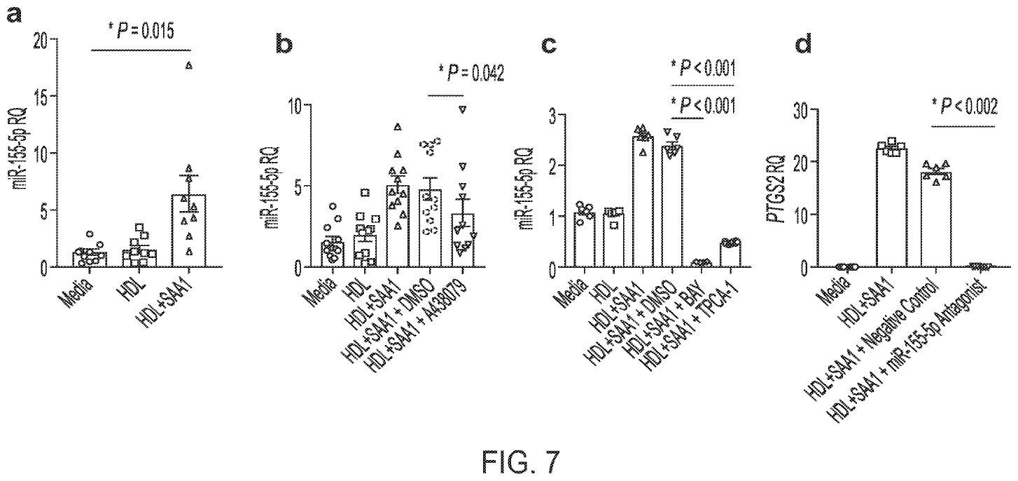

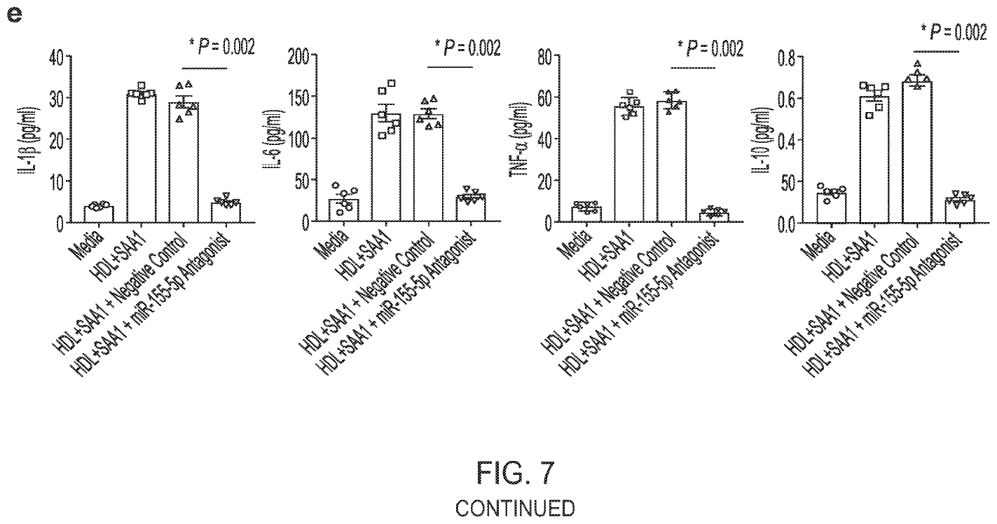

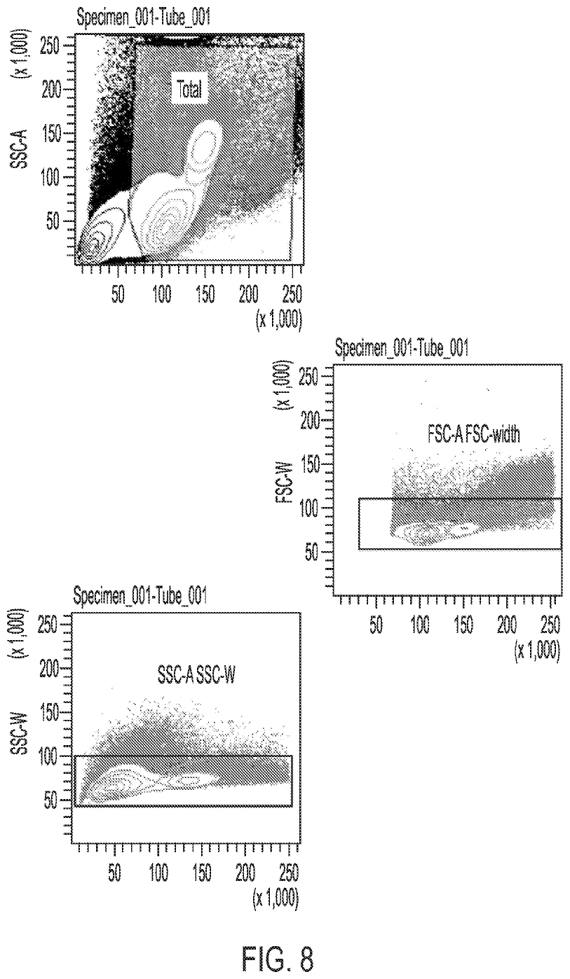

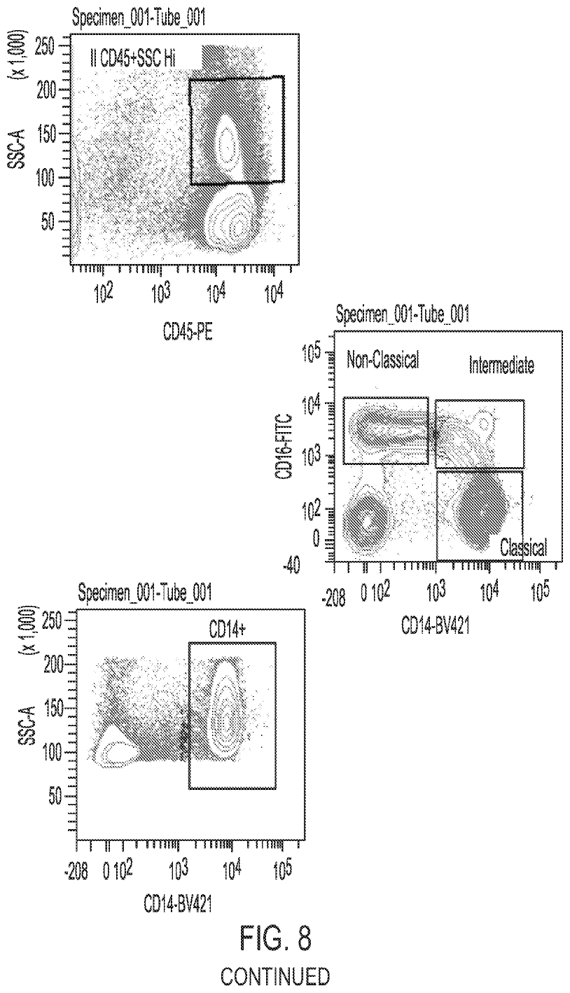

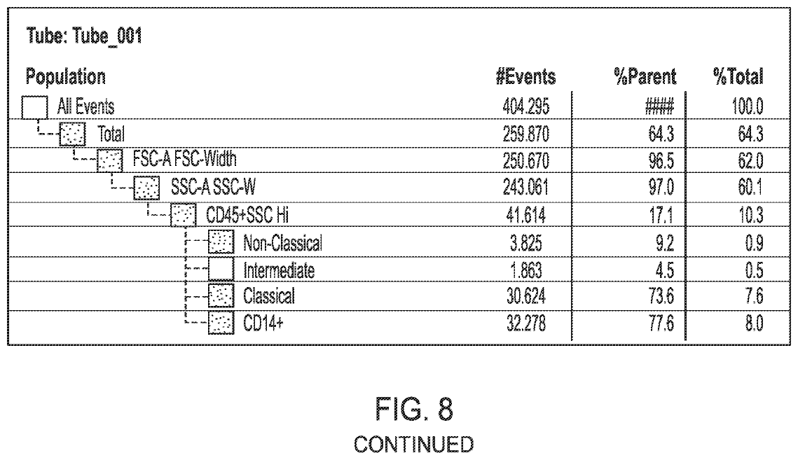

The RNA-seq analysis (FIG. 4a) also identified the increased expression of MIR155HG (miR-155 host gene), which is a microRNA expressed in monocytes that promotes inflammation via several mechanisms, including the direct binding of miR-155 to the 3′ untranslated region of PTGS2 mRNA. miR-155 thereby increases the stability of PTGS2 mRNA transcripts, which up-regulates both PTGS2 mRNA and COX-2 protein levels. Furthermore, lungs from miR-155^{-/-} mice challenged with cockroach extract have reductions in both COX-2 expression and eosinophilic inflammation. Since SAA is not known to mediate its effects via miR-155, the study investigated the role of miR-155 in HDL+SAA1-induced cytokine secretion by CD14⁺ monocytes. First, it was confirmed by qRT-PCR that HDL+SAA1 increased miR-155-5p levels in CD14⁺ monocytes (FIG. 7a). Second, HDL+SAA1-mediated increases in miR-155-5p were significantly reduced by the P2X7R antagonist, A438079 (FIG. 7b). The human MIR155HG promoter contains a NF-κB-responsive site that binds NF-κB p50/p65 heterodimers and increases expression of miR-155. Consistent with this, we show that inhibitors of NF-κB signaling, BAY 11-7082 and TPCA1, attenuated HDL+SAA1-mediated increases in miR-155-5p (FIG. 7c). Lastly, a miR-155-5p antagonist suppressed HDL+SAA1-mediated increases in PTGS2 mRNA (FIG. 7d), as well as the secretion of IL-1β, IL-6, TNF-α, and IL-10 (FIG. 7e). Collectively, this identifies a role for miR-155 acting downstream of HDL+SAA1, P2X7R, and NF-κB, to up-regulate COX-2 expression, with resultant increased cytokine secretion by CD14⁺ monocytes.

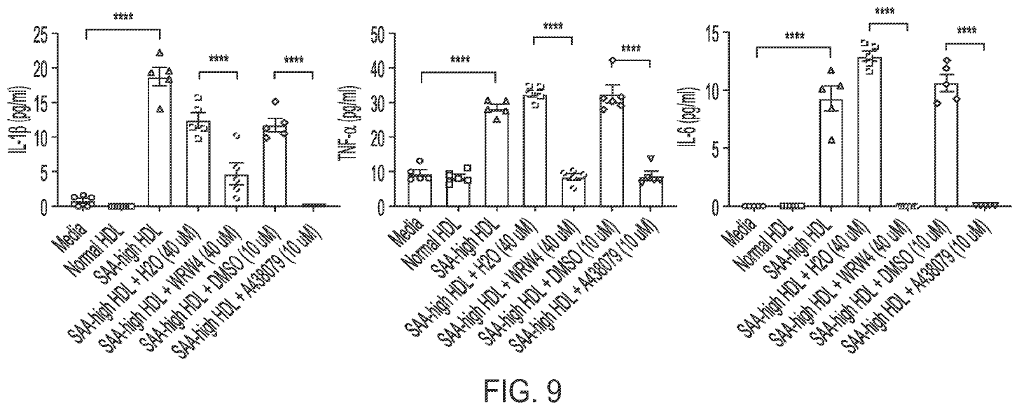

Stimulation of THP-1 monocytes with endogenous HDL from SAA-high asthmatics and suppression of cytokine secretion were further studied. HDL (4 μg of protein/ml) isolated from plasma from a SAA-high asthmatic (SAA-high HDL) and from a healthy non-asthmatic subject (Normal HDL) were used to stimulate THP-1 monocytes for 24 h. THP-1 monocytes were pre-treated with the FPR2 antagonist, WRW4 or water (40 μM) as the vehicle control, or A438079 or DMSO (10 μM) as the vehicle control, for 1 h prior to stimulation with normal or SAA-high HDL for 24 h. One-way ANOVA with Sidak's multiple comparisons test. ****P<0.0001. As illustrated in FIG. 9, IL-1β, IL-6, and TNF-α secretion by THP-1 monocytes could be inhibited by both the FPR2 antagonist, WRW4, as well as the P2X7R antagonist, A438079.

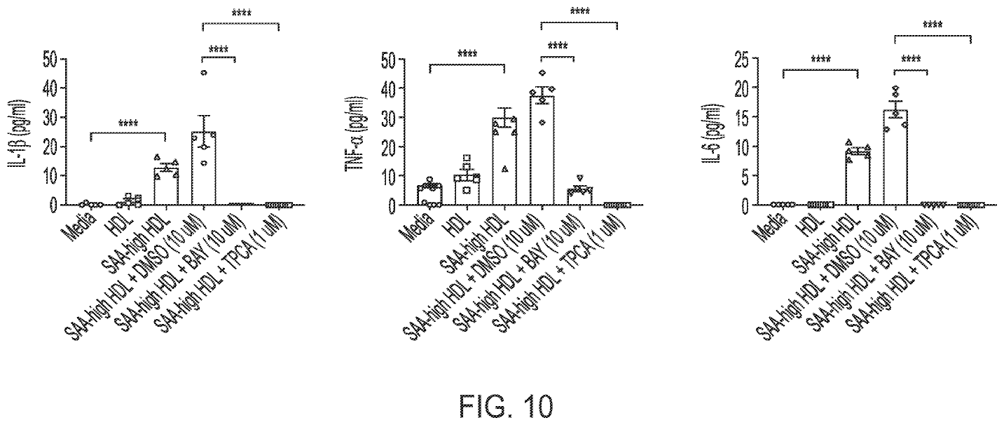

As shown in FIG. 10, inhibitors of NF-κB signaling pathways, BAY 11-7082 and TPCA1, abrogate the ability of endogenous SAA-high HDL to induce cytokine secretion by THP-1 monocytes. HDL (4 μs of protein/ml) isolated from plasma from a SAA-high asthmatic (SAA-high HDL) and from a healthy non-asthmatic subject (Normal HDL) were used to stimulate THP-1 monocytes for 24 h. THP-1 monocytes were pre-treated with BAY-11-7082 (10 TPCA-1 (1 μM) or DMSO (10 μM) as the vehicle control for 1 h, as indicated, prior to stimulation with normal or SAA-high HDL for 24 h. One-way ANOVA with Sidak's multiple comparisons test. ****P<0.0001.

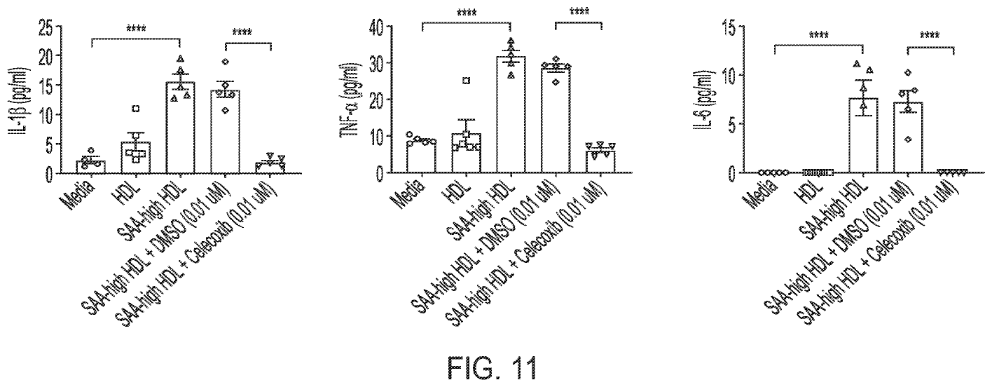

FIG. 11 further shows that Celecoxib (10 nM) inhibits endogenous SAA-high HDL-induced increases in IL-1β, IL-6, and TNF-α secretion by THP-1 monocytes. HDL (4 μg of protein/ml) isolated from plasma from a SAA-high asthmatic (SAA-high HDL) and from a healthy non-asthmatic subject (Normal HDL) were used to stimulate THP-1 monocytes for 24 h. A. THP-1 monocytes were pre-treated with celecoxib or DMSO (0.01 μM) as the vehicle control for 1 h, as indicated, prior to stimulation with normal or SAA-high HDL for 24 h. One-way ANOVA with Sidak's multiple comparisons test. ****P<0.0001.

It will be appreciated by persons skilled in the art that the invention described herein is not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific agent, or a step of the method, and may result from a different combination of described agent or step, or that other undescribed alternate embodiments may be available for an agent or method, is not to be considered a disclaimer of those alternate embodiments. It will be appreciated that many of those undescribed embodiments are within the literal scope of the following claims, and others are equivalent.

1. A method of treating an asthmatic subject, comprising:
determining serum level of serum amyloid A (SAA) in the subject;
(ii) comparing the serum level with a pre-determined threshold; and
(iii) administering to the subject a therapeutically effective amount of a COX-2 inhibitor if the serum level is greater than or equal to the threshold.

2. The method of claim 1, wherein the threshold is 95^{th} percentile of non-asthmatic subjects.

3. The method of claim 1, wherein the threshold is about 108 μg/ml.

4. The method of claim 1, wherein the threshold is 90^{th} percentile of asthmatic subjects.

5. The method of claim 1, wherein the COX-2 inhibitors is an agent selected from the group consisting of acetylsalicylic acid (aspirin), 2-(4-isobutylphenyl)propanoic acid (ibuprofen), N-(4-hydroxyphenyl)ethanamide (paracetamol), (S)-6-methoxy-α-methyl-2-naphthaleneacetic acid (naproxen), 2-[(2,6-dichlorophenyl)amino] benzeneacetic acid (diclofenac), 4-[5-(4-methylphenyl)-3-(trifluoromethyl)-1H-pyrazol-1-yl] benzenesulfonamide (celecoxib), 4-[4-(methylsulfonyl)phenyl]-3-phenyl-2(5H)-furanone (rofecoxib), and 4-(5-Methyl-3-phenylisoxazol-4-yl)benzolsulfonamid (valdecoxib).

6. The method of claim 1, further comprising administering to the subject an additional agent for treating asthma.

7. The method of claim 1, further comprising determining the subject as having higher than normal body-mass index (BMI), higher than normal serum C-reactive protein, or lower than normal serum IgE.

8. A method of reducing inflammation associated with abnormal level of serum amyloid A (SAA) in a subject, comprising:
determining serum level of serum amyloid A (SAA) in the subject;
(ii) comparing the serum level with a pre-determined threshold; and
(iii) administering to the subject a therapeutically effective amount of a COX-2 inhibitor if the serum level is greater than or equal to the threshold.

9. The method of claim 8, wherein the subject has been diagnosed to have asthma.

10. The method of claim 8, wherein the threshold is 95^{th} percentile of SAA levels from non-asthmatic subjects.

11. The method of claim 8, wherein the threshold is about 108 μg/ml.

12. The method of claim 8, wherein the threshold is 90^{th} percentile of SAA levels from asthmatic subjects.

13. The method of claim 8, further comprising determining the subject as having higher than normal level of cytokine.

14. The method of claim 8, further comprising determining the subject as having higher than normal level of one or more cytokines selected from the group consisting of IL-6, IL-10, and TNF-α.

15. The method of claim 8, wherein the COX-2 inhibitors is an agent selected from the group consisting of acetylsalicylic acid (aspirin), 2-(4-isobutylphenyl)propanoic acid (ibuprofen), N-(4-hydroxyphenyl)ethanamide (paracetamol), (S)-6-methoxy-α-methyl-2-naphthaleneacetic acid (naproxen), 2-[(2,6-dichlorophenyl)amino] benzeneacetic acid (diclofenac), 4-[5-(4-methylphenyl)-3-(trifluoromethyl)-1H-pyrazol-1-yl] benzenesulfonamide (celecoxib), 4-[4-(methylsulfonyl)phenyl]-3-phenyl-2(5H)-furanone (rofecoxib), and 4-(5-Methyl-3-phenylisoxazol-4-yl)benzolsulfonamid (valdecoxib).

16. The method of claim 8, further comprising administering to the subject one or more agents selected from the group consisting of an FPR2 inhibitor, a P2X7R inhibitor and an NF-κB antagonist.

17. The method of claim 8, further comprising determining the subject as having higher than normal body-mass index (BMI), higher than normal serum C-reactive protein, or lower than normal serum IgE.

18. A method to identify a asthma subject suitable for treatment with COX-2 inhibitor, comprising:
determining serum level of serum amyloid A (SAA) in the subject;
(ii) comparing the serum level with a pre-determined threshold; and
(iii) identifying the subject as suitable for treatment with COX-2 inhibitor if the serum SAA level in the subject is greater than or equal to the threshold.

19. The method of claim 18, wherein the threshold is 95^{th} percentile of SAA levels from non-asthmatic subjects.

20. The method of claim 18, wherein the threshold is about 108 μg/ml.

21. The method of claim 18, wherein the threshold is 90^{th} percentile of SAA levels from asthmatic subjects.

22. The method of claim 18, further comprising determining the subject as having higher than normal level of one or more cytokines selected from the group consisting of IL-6, IL-10, and TNF-α.

23. A method of treating asthma in a subject in need thereof, wherein the subject has a serum SAA level of equal or greater than about 100 μg/ml, comprising administering to the subject a therapeutically effective amount of a COX-2 inhibitor.

24. The method of claim 23, further comprising, prior to administering the COX-2 inhibitor, determining the subject as having higher than normal level of one or more cytokines selected from the group consisting of IL-6, IL-10, and TNF-α.

25. The method of claim 23, wherein the COX-2 inhibitors is an agent selected from the group consisting of acetylsalicylic acid (aspirin), 2-(4-isobutylphenyl)propanoic acid (ibuprofen), N-(4-hydroxyphenyl)ethanamide (paracetamol), (S)-6-methoxy-α-methyl-2-naphthaleneacetic acid (naproxen), 2-[(2,6-dichlorophenyl)amino] benzeneacetic acid (diclofenac), 4-[5-(4-methylphenyl)-3-(trifluoromethyl)-1H-pyrazol-1-yl] benzenesulfonamide (celecoxib), 4-[4-(methylsulfonyl)phenyl]-3-phenyl-2(5H)-furanone (rofecoxib), and 4-(5-Methyl-3-phenylisoxazol-4-yl)benzolsulfonamid (valdecoxib).

26. The method of claim 23, further comprising administering to the subject an additional agent for treating asthma.

27. The method of claim 23, further comprising, prior to administering the COX-2 inhibitor, determining the subject as having higher than normal body-mass index (BMI), higher than normal serum C-reactive protein, or lower than normal serum IgE.

28. The method of claim 23, further comprising administering to the subject one or more agents selected from the group consisting of an FPR2 inhibitor, a P2X7R inhibitor and an NF-κB antagonist.
